SEQUENCE LISTING (33 sequences)

SEQ ID NO: 1 - DNA, 423 bp, Malus domestica
aattcccccgtttctctctttccctctcgccgctctctcctccatctccgtccccaaacgctaggtgtgtctccgccaaaccactagatatgggaggcagaggagttatcggggatcgatggtccatgaggattctctgggcgtgtgcaattggaagtgctgtcagcctatatatggttgctgtggacagacaactaaagaacagggaacgagcgctggccgaagagttaaaggccatggaggcagaatcaggcagcggtgagattgtttgactgttgataaattataggcaataactagcttagagctttctagatttcccaagttcgcatctgtcattttggccattgtgaggattatgtaaaatgttgtttgttgttcccattcggaatcaagttttagggggtttaccccgctgacaag

SEQ ID NO: 2 - protein, 60 aa, Malus domestica
MGGRGVIGDRWSMRILWACAIGSAVSLYMVAVDRQLKNRERALAEELKAMEAESGSGEIV

SEQ ID NO: 3 - DNA, 580 bp, Arabidopsis thaliana
aattgttttaaaattacaaattagtccgttcttttattcccgtactcgttccttcttcttcttcttcctctcatcgtcattttctcgattctcactcttccggtcaccgactaattctgaataaggtttatcaaaagaataagaataagtggataaaaagctagctttgaaagagttattgcagagaaaaaaaatgggatcgagagggattatcaacgataagtggtcaatgaggattctatggggttgtgctatcggaagtgctattggtttatacatggttgctgtagagagacaaactcagaacagggctcgtgctatggctgagagtttgagagctgctgaatcacaaggtgatggtgataatgtctaatatctaccaagtagtgctcagttgaatactctcagttgagtttttttttttggtgtttgtttttgttataatgacttcttctgccaagatggtgttgatgtagtttcttttttgcaaataatcgtaataaggtttcgaaacttggagagttgaagttgctgaacatacgatttgtgttatcgcaaaaaaagttatttcttatgcctg

SEQ ID NO: 4 - protein, 59 aa, Arabidopsis thaliana
MGSRGIINDKWSMRILWGCAIGSAIGLYMVAVERQTQNRARAMAESLRAAESQGDGDNV

SEQ ID NO: 5 - DNA, 498 bp, Oryza sativa
actcggaggctgcggcccgcacggcgaacggagcggcggcgcagctcgcgcgatcaatcgtcggcggcagcggcggcggcggcggcgggatggggctcggggggcgaggcgtggtgggggagaggtggtcgcagcgcgtcctctggctctgcgccatcggcagcgccgtgagcctgtactacgtggcggtggagaggcaggcgcagaaccgcgcgcgggcggtggccgaggggctcaaggccctcgacggcgccggcgccggagaggacgtgtgacttcgctgtgtgctggagaggtgatcccggcctgtgtagagacggcctctctgttcgagctcgaaacgagttatatttttgcttaccttgtttcttgtttcatgaaattttcgcaataataatgtactagtaattgccccctttgtcattgcgataactggattacaatttgcgatatgggagccagaaatgatggccgaaatgaatgtcatctgtttgttct

SEQ ID NO: 6 - protein, 61 aa, Oryza sativa
MGLGGRGVVGERWSQRVLWLCAIGSAVSLYYVAVERQAQNRARAVAEGLKALDGAGAGEDV

SEQ ID NO: 7 - DNA, 430 bp, Oryza sativa
actcggaggctgcggcccgcacggcgaacggagcggcggcgcagctcgcgcgatcaatcgccgcctaatcgcagtggtttgatccggcggcgtcgccgcgtgtgcaggcctgtactacgtggcggtggagaggcaggcgcagaaccgcgcgcgggcggtggccgaggggctcaaggccctcgacggcgccggcgccggagaggacgtgtgacttcgctgtgtgctggagaggtgatcccggcctgtgtagagacggcctctctgttcgagctcgaaacgagttatatttttgcttaccttgtttcttgtttcatgaaattttcgcaataataatgtactagtaattgccccctttgtcattgcgataactggattacaatttgcgatatgggagccagaaatgatggccgaaatgaatgtcatctgtttg

SEQ ID NO: 8 - DNA, 23 bp, Artificial
  Primer
ttagatatccacataacatgtgc

SEQ ID NO: 9 - DNA, 22 bp, Artificial
  Primer
ttcacaaacatagcatgacagg

SEQ ID NO: 10 - DNA, 22 bp, Artificial
  Primer
tggttcacgtagtgggccatcg

SEQ ID NO: 11 - DNA, 22 bp, Artificial
  Primer
acagcacttccaattgcacacg

SEQ ID NO: 12 - DNA, 22 bp, Artificial
  Primer
ctttagttgtctgtccacagca

SEQ ID NO: 13 - DNA, 28 bp, Artificial
  Primer
aggaattcatgagtctgaatacaagtgc

SEQ ID NO: 14 - DNA, 27 bp, Artificial
  Primer
gcggatccaagcttaagccgcgcccag

SEQ ID NO: 15 - DNA, 28 bp, Artificial
  Primer
cggaattcaacgataagtggtcaatgag

SEQ ID NO: 16 - DNA, 32 bp, Artificial
  Primer
cgggatccttagacattatcaccatcaccttg

SEQ ID NO: 17 - DNA, 32 bp, Artificial
  Primer
gccgctcgaggtattcaactgagcactacttg

SEQ ID NO: 18 - DNA, 32 bp, Artificial
  Primer
cgggatccttacttggctttgttgaactgctc

SEQ ID NO: 19 - DNA, 32 bp, Artificial
  Primer
cgggatccttagaactgaccgatttccttcgc

SEQ ID NO: 20 - DNA, 31 bp, Artificial
  Primer
cgggatccttacaggttttgcagccctttgc

SEQ ID NO: 21 - DNA, 33 bp, Artificial
  Primer
cgggatccttaataggcgttctgctcgccttcg

SEQ ID NO: 22 - DNA, 33 bp, Artificial
  Primer
cgggatccttaggacgttgagttaatacccagc

SEQ ID NO: 23 - DNA, 26 bp, Artificial
  Primer
cggaattcgataccgtcaatcagctg

SEQ ID NO: 24 - DNA, 26 bp, Artificial
  Primer
cggaattcctgaacgatatgttaggc

SEQ ID NO: 25 - DNA, 29 bp, artificial
  Primer
cggaattccagctgctgaagatgttcagc

SEQ ID NO: 26 - DNA, 30 bp, Artificial
  Primer
cggaattcaatgctggcacgggtcttgacg

SEQ ID NO: 27 - DNA, 182 bp, Artificial
  Sense sequence cloned into HIPM silencing construct
atgggaggcagaggagttatcggggatcgatggtccatgaggattctctgggcgtgtgcaattggaagtgctgtcagcctatatatggttgctgtggacagacaactaaagaacagggaacgagcgctggccgaagagttaaaggccatggaggcagaatcaggcagcggtgagattgtttg

SEQ ID NO: 28 - DNA, 178 bp, Artificial
  Sense sequence cloned into AtHIPM silencing construct
atgggatcgagagggattatcaacgataagtggtcaatgaggattctatggggttgtgctatcggaagtgctattggtttatacatggttgctgtagagagacaaactcagaacagggctcgtgctatggctgagagtttgagagctgctgaatcacaaggtgatggtgataatgtct

SEQ ID NO: 29 - DNA, 430 bp, Artificial; identical to SEQ ID NO: 7
  Sense sequence cloned into OsHIPM silencing construct

SEQ ID NO: 30 - DNA, 312 bp, Artificial
  Sense sequence cloned into OsHIPM silencing construct
gtggcggtggagaggcaggcgcagaaccgcgcgcgggcggtggccgaggggctcaaggccctcgacggcgccggcgccggagaggacgtgtgacttcgctgtgtgctggagaggtgatcccggcctgtgtagagacggcctctctgttcgagctcgaaacgagttatatttttgcttaccttgtttcttgtttcatgaaattttcgcaataataatgtactagtaattgccccctttgtcattgcgataactggattacaatttgcgatatgggagccagaaatgatggccgaaatgaatgtcatctgtttg

SEQ ID NO: 31 - DNA, 183 bp, Artificial
  Sequence cloned into HIPM overexpression construct
atgggaggcagaggagttatcggggatcgatggtccatgaggattctctgggcgtgtgcaattggaagtgctgtcagcctatatatggttgctgtggacagacaactaaagaacagggaacgagcgctggccgaagagttaaaggccatggaggcagaatcaggcagcggtgagattgtttga

SEQ ID NO: 32 - DNA, 180 bp, Artificial
  Sequence cloned into AtHIPM overexpression construct
atgggatcgagagggattatcaacgataagtggtcaatgaggattctatggggttgtgctatcggaagtgctattggtttatacatggttgctgtagagagacaaactcagaacagggctcgtgctatggctgagagtttgagagctgctgaatcacaaggtgatggtgataatgtctaa

SEQ ID NO: 33 - DNA, 188 bp, Artificial
  Sequence cloned into OsHIPM overexpression construct
atggggctcggggggcgaggcgtggtgggggagaggtggtcgcagcgcgtcctctggctctgcgccatcggcagcgccgtgagcctgtactacgtggcggtggagaggcaggcgcagaaccgcgcgcgggcggtggccgaggggctcaaggccctcgacggcgccggcgccggagaggacgtgtgact

(19) United States
(12) Patent Application Publication
(10) Pub. No.: US 2009/0044296 A1
(43) Pub. Date: Feb. 12, 2009

(54) HRPN INTERACTORS AND USES THEREOF

(75) Inventors: Steven V. BEER, Ithaca, NY (US); Ray J. WU, Ithaca, NY (US); Chang-Sik OH, Ithaca, NY (US)

Correspondence Address: NIXON PEABODY LLP - PATENT GROUP, 1100 CLINTON SQUARE, ROCHESTER, NY, 14604, US

(73) Assignee: CORNELL RESEARCH FOUNDATION, INC., Ithaca, NY (US)

(21) Appl. No.: 11/835,038

(22) Filed: Aug. 7, 2007

(51) Int. Cl. A01H 1/00; A01H 5/00; C07H 21/00; C07K 14/00; C12N 1/00; C12N 15/63

(52) U.S. Cl. 800/279; 435/243; 435/320.1; 530/350; 536/23.1; 536/24.1; 800/290; 800/295

(57) ABSTRACT

The present invention relates to nucleic acid molecules configured to increase or decrease expression of a nucleic acid molecule that encodes a HrpN-interacting protein; nucleic acid constructs that include these nucleic acid molecules; host cells, transgenic plants, and transgenic plant seeds transformed thereby; and methods of increasing plant growth or imparting disease resistance to plants. Also disclosed are an isolated HIPM nucleic acid molecule and an isolated HIPM protein or polypeptide.

The subject matter of this application was made with support from the United States Government under USDA CSREES Special Grant 2003-34367-13158. The U.S. Government may have certain rights in this invention.

### FIELD OF THE INVENTION

The present invention relates to HrpN-interactors and uses thereof.

### BACKGROUND OF THE INVENTION

Harpins are proteins from Gram-negative plant-pathogenic bacteria with the following distinctive characteristics: they are heat-stable and glycine-rich, and have no cysteine and few aromatic amino acids. Since HrpN of E. amylovora was characterized as the first cell-free elicitor of the hypersensitive response in plants (Wei et al., “Harpin, Elicitor of the Hypersensitive Response Produced by the Plant Pathogen Erwinia amylovora,” Science 257:85-8 (1992)), several other harpins have been characterized from various Gram-negative plant-pathogenic bacteria: HrpN and HrpW of Erwinia spp.; HrpZ; HrpW; HopPtoP; HopPmaH_{Pto} of Pseudomonas syringae; PopA1 of Ralstonia solanacearum; and HpaG and its orthologs of Xanthomonas campestris like XopA (He et al., “Pseudomonas syringae pv. syringae Harpin_{Pss}: A Protein That Is Secreted Via the Hrp Pathway and Elicits the Hypersensitive Response in Plants,” Cell 73:1255-66 (1993); Arlat et al., “PopA1, A Protein Which Induces a Hypersensitivity-like Response on Specific Petunia Genotypes, Is Secreted Via the Hrp Pathway of Pseudomonas solanacearum,” EMBO J. 13(3):543-53 (1994); Bauer et al., “Erwinia chrysanthemi Harpin_{Ech}: An Elicitor of the Hypersensitive Response that Contributes to Soft-rot Pathogenesis,” Mol. Plant-Microbe Interact. 8:484-91 (1995); Charkowski et al., “The Pseudomonas syringae pv. tomato HrpW Protein Has Domains Similar to Harpins and Pectate Lyases and Can Elicit the Plant Hypersensitive Response and Bind to Pectate,” J. Bacteriol. 180:5211-7 (1998); Kim & Beer, “HrpW of Erwinia amylovora, a New Harpin That Contains a Domain Homologous to Pectate Lyases of a Distinct Class,” J. Bacteriol. 180:5203-10 (1998); Kim et al., “Mutational Analysis of Xanthomonas Harpin HpaG Identifies a Key Functional Region That Elicits the Hypersensitive Response in Nonhost Plants,” J. Bacteriol. 186:6239-47 (2004); Ramos, “Hrp Proteins and Harpins: Defining Their Roles in the Type III Protein Secretion System in Pseudomonas syringae,” (Cornell Univ. 2004)). Harpins are secreted through the Hrp type III secretion system (“T3SS”) like avirulence (“Avr”) proteins of plant pathogenic bacteria, which directly or indirectly interact with corresponding resistance proteins (Alfano & Collmer, “Type III Secretion System Effector Proteins: Double Agents in Bacterial Disease and Plant Defense,” Annu. Rev. Phytopathol. 42:385-414 (2004)). However, unlike Avr proteins, which mostly are delivered to the plant cytoplasm, harpins are located to the plant apoplast.

All harpins except XopA induce a hypersensitive response in tobacco following infiltration of the intercellular spaces of leaf panels. Mutational analysis of HpaG showed that a 12 amino acid region between Leu39 and Leu50 is critical to hypersensitive response elicitation in tobacco. Also, site-directed mutagenesis of Phe48 to Leu48 in XopA restored its hypersensitive response-eliciting activity (Kim et al., “Mutational Analysis of Xanthomonas Harpin HpaG Identifies a Key Functional Region That Elicits the Hypersensitive Response in Nonhost Plants,” J. Bacteriol. 186:6239-47 (2004)). Although the mechanisms of hypersensitive response elicitation by harpins are not understood, several suggestions have been made that are supported by some experimental evidence. First, harpins may disturb membrane physiology and result in cell death. Both HrpZ and PopA have pore-forming activity in artificial membranes (Racape et al., “Ca²⁺-dependent Lipid Binding and Membrane Integration of PopA, a Harpin-like Elicitor of the Hypersensitive Response in Tobacco,” Mol. Microbiol. 58:1406-20 (2005); Lee et al., “HrpZ(Psph) from the Plant Pathogen Pseudomonas syringae pv. phaseolicola Binds to Lipid Bilayers and Forms an Ion-conducting Pore In Vitro,” Proc. Nat'l Acad. Sci. USA 98:289-94 (2001)). In addition, HrpN induces ion leakage by stimulating plasma ion channels (El-Maarouf et al., “Harpin, a Hypersensitive Response Elicitor from Erwinia amylovora, Regulates Ion Channel Activities in Arabidopsis thaliana Suspension Cells,” FEBS Lett. 497:82-4 (2001)). Secondly, harpins may indirectly disturb mitochondrial functions and induce mitochondria-dependent programmed cell death in plants. Treatment of Arabidopsis cells with HrpZ induces rapid release of cytochrome C from mitochondria to the cytosol, and reactive oxygen species accumulate (Krause & Durner, “Harpin Inactivates Mitochondria in Arabidopsis Suspension Cells,” Mol. Plant-Microbe Interact. 17:131-9 (2004)). There is also evidence that HrpN may inhibit ATP synthesis by reducing mitochondrial electron transport in tobacco cells (Xie & Chen, “Harpin-induced Hypersensitive Cell Death Is Associated with Altered Mitochondrial Functions in Tobacco Cells,” Mol. Plant-Microbe Interact. 13:183-90 (2000)). Thirdly, harpins may need signal transduction pathways to induce a hypersensitive response. AvrPtoB, a known suppressor of the hypersensitive response, suppresses HrpN-dependent hypersensitive response in tobacco (Oh et al., “The Hrp Pathogenicity Island of Erwinia amylovora and the Identification of Three Novel Genes Required for Systemic Infection,” Mol. Plant Pathol. 6:125-38 (2005)). HrpZ induces hypersensitive response-related genes like Hin1 and activates protein kinases such as AtMPK6 in Arabidopsis and its ortholog SIPK in tobacco (Gopalan et al., “Hrp Gene-dependent Induction of hin1: A Plant Gene Activated Rapidly by Both Harpins and the avrPto Gene-mediated Signal,” Plant J. 10:591-600 (1996); Zhang & Klessig, “Pathogen-induced MAP Kinases in Tobacco,” Results Probl. Cell Differ. 27:65-84 (2000); Desikan et al., “Harpin Induces Activation of the Arabidopsis Mitogen-activated Protein Kinases AtMPK4 and AtMPK6,” Plant Physiol. 126:1579-87 (2001)). Lastly, harpins may induce hypersensitive response from outside plant cells. Extracellularly targeted HrpN and HrpZ induce a hypersensitive response in tobacco (Tampakaki & Panopoulos, “Elicitation of Hypersensitive Cell Death by Extracellularly Targeted HrpZ_{Psph} Produced in Planta,” Mol. Plant-Microbe Interact. 13:1366-74 (2000); Oh, “Characterization of HrpN-interacting Proteins from Plants, the Hrp Pathogenicity Island of Erwinia amylovora, and its Proteins That Affect the Hypersensitive Response,” (Ph.D. thesis, Cornell University 2005)), while the same proteins targeted to the cytoplasm do not.

In addition to hypersensitive response elicitation, some harpins reportedly have virulence functions in host plants. Mutation of hpaG by transposon insertion or mutation of its ortholog xopA by deletion, results in reduced symptoms and reduced bacterial growth in host plants (Noel et al., “Two Novel Type III-secreted Proteins of Xanthomonas campestris pv. vesicatoria are Encoded Within the hrp Pathogenicity Island,” J. Bacteriol. 184:1340-8 (2002); Kim et al., “Characterization of the Xanthomonas axonopodis pv. glycines Hrp Pathogenicity Island,” J. Bacteriol. 185:3155-66 (2003)). The most striking example of function in virulence is the hrpN gene of E. amylovora. Mutation of hrpN results in drastically reduced virulence (Barny, “Erwinia amylovora hrpN Mutants, Blocked in Harpin Synthesis, Express a Reduced Virulence on Host Plants and Elicit Variable Hypersensitive Reactions on Tobacco,” Eur. J. Plant Pathol. 101:333-40 (1995)). Consistently, a mutant of E. amylovora strain Ea273, in which the hrpN gene had been substantially deleted, caused less than 3% of apple shoot length to blight, versus approximately 80% blighted by the wild-type strain. However, why plant-pathogenic bacteria produce harpins and why host plants apparently do not recognize harpins for induction of defense responses remain to be determined.

Interestingly, when plants are sprayed with HrpN, several beneficial effects result: induction of resistance to pathogens inducing systemic acquired resistance, induction of resistance to aphids, and enhancement of plant growth. First, HrpN of E. amylovora induces systemic acquired resistance, resulting in resistance to pathogens in Arabidopsis (Dong et al., “Harpin Induces Disease Resistance in Arabidopsis Through the Systemic Acquired Resistance Pathway Mediated by Salicylic Acid and the NIM1 Gene,” Plant J. 20:207-15 (1999)). Systemic acquired resistance is mediated by salicylic acid and NPR1/NIM1, which are key components. HrpN-induced pathogen resistance also requires NDR1 and EDS1 genes, which are involved in signal transduction pathways for the resistance protein-dependent hypersensitive response (Peng et al., “Harpin-elicited Hypersensitive Cell Death and Pathogen Resistance Requires the NDR1 and EDS1 Genes,” Physiol. Mol. Plant Pathol. 62:317-26 (2003)). Secondly, HrpN increases resistance to aphids in Arabidopsis. The total number of aphids on HrpN-treated Arabidopsis was one third of those on buffer-treated Arabidopsis, 7 days after infestation (Dong et al., “Downstream Divergence of the Ethylene Signaling Pathway for Harpin-stimulated Arabidopsis Growth and Insect Defense,” Plant Physiol. 136:3628-38 (2004)). Aphid numbers were significantly reduced in the wild-type, npr1-1, and jar1-1 mutants by treatment with HrpN, but not in both etr1-1 and ein2-1 mutants, indicating that the ethylene signaling pathway may be involved in aphid resistance by treatment with HrpN (Dong et al., “Downstream Divergence of the Ethylene Signaling Pathway for Harpin-stimulated Arabidopsis Growth and Insect Defense,” Plant Physiol. 136:3628-38 (2004)). Lastly, HrpN promotes plant growth and increases plant productivity in plants. Plant growth is enhanced in both npr1-1 and jar1-1 mutants of Arabidopsis, but not in etr1-1 and ein5-1 mutants, indicating that the ethylene-signaling pathway is involved in enhanced plant growth responding to treatment with HrpN (Dong et al., “Downstream Divergence of the Ethylene Signaling Pathway for Harpin-stimulated Arabidopsis Growth and Insect Defense,” Plant Physiol. 136:3628-38 (2004)). However, how the harpin signal is perceived and transmitted to these multiple signaling pathways in planta remains to be determined.

The present invention is directed to overcoming these and other deficiencies in the art.

### SUMMARY OF THE INVENTION

The present invention relates to a nucleic acid molecule configured to increase or decrease expression of a nucleic acid molecule that encodes a HrpN-interacting protein. The HrpN-interacting protein is (i) a protein having an amino acid sequence selected from the group of SEQ ID NO: 2, SEQ ID NO: 4, and SEQ ID NO: 6; (ii) a protein encoded by a nucleotide sequence of SEQ ID NO: 1, SEQ ID NO: 3, and SEQ ID NO: 5; and (iii) a protein at least 90% homologous and/or identical to the protein of (i) or (ii).

Another aspect of the present invention relates to a nucleic acid construct that includes the nucleic acid molecule of the present invention, a 5′ promoter sequence, and a 3′ terminator sequence, operatively coupled to permit transcription of the nucleic acid molecule.

A further aspect of the present invention relates to a method of increasing or decreasing plant growth. This method involves providing a transgenic plant or plant seed transformed with a nucleic acid construct of the present invention and growing the transgenic plant or a transgenic plant grown from the transgenic plant seed under conditions effective to increase plant growth compared to non-transgenic plants.

Another aspect of the present invention relates to a method of imparting disease resistance to plants. This method involves providing a transgenic plant or plant seed transformed with a nucleic acid construct of the present invention and growing the transgenic plant or a transgenic plant grown from the transgenic plant seed under conditions effective to impart disease resistance to the plant compared to non-transgenic plants.

The present invention also relates to an isolated nucleic acid molecule comprising bases 90 to 269 of the nucleotide sequence of SEQ ID NO: 1.

A further aspect of the present invention relates to an isolated protein or polypeptide that includes the amino acid sequence of SEQ ID NO: 2.

As disclosed herein, it was hypothesized that harpins interact with plant proteins to increase susceptibility in host plants and also to induce multiple signaling pathways for beneficial effects in plants like Arabidopsis. As a first step, HrpN-interacting proteins from apple, an important host, were identified using a yeast two-hybrid assay. One protein, designated HIPM, was found. Based on the amino acid sequence of HIPM, an ortholog in Arabidopsis (AtHIPM) and an ortholog in the Nipponbare cultivar of rice (OsHIPM-N) were found. Both HIPM and AtHIPM interacted with HrpN in yeast and in vitro. Both HIPM and AtHIPM have functional signal peptides and associate, in clusters, with plasma membranes. In addition, it was found that AtHIPM is needed for Arabidopsis to exhibit enhanced growth in response to HrpN and functions as a negative regulator of plant growth. Domain analysis of OsHIPM-N using its amino acid sequence showed that it has a putative signal peptide and a putative transmembrane domain like HIPM, indicating that OsHIPM-N functions similarly to HIPM and AtHIPM.

### BRIEF DESCRIPTION OF THE DRAWINGS

FIG. 1 shows the nucleotide (SEQ ID NO: 1) and amino acid (SEQ ID NO: 2) sequences of the apple HIPM gene. The start codon of the HIPM gene, ATG, is underlined, and the asterisk indicates the stop codon. The nucleotide sequence of both the 5′-UTR upstream of the start codon and the 3′-UTR downstream of the stop codon are shown.

FIG. 2 shows the nucleotide (SEQ ID NO: 3) and amino acid (SEQ ID NO: 4) sequences of the AtHIPM gene of A. thaliana. The start codon of the AtHIPM gene, ATG, is underlined, and the asterisk indicates the stop codon. The nucleotide sequence of both the 5′-UTR upstream of the start codon and the 3′-UTR downstream of the stop codon are shown.

FIG. 3 shows the nucleotide (SEQ ID NO: 5) and amino acid (SEQ ID NO: 6) sequences of the rice OsHIPM gene from the Nipponbare cultivar (“OsHIPM-N”). The start codon of the OsHIPM-N gene, ATG, is underlined, and the asterisk indicates the stop codon. The nucleotide sequence of both the 5′-UTR upstream of the start codon and the 3′-UTR downstream of the stop codon are shown.

FIG. 4 is a sequence alignment comparing the OsHIPM gene of the Jefferson cultivar (“OsHIPM-J”) (SEQ ID NO: 7) with the OsHIPM gene of the Nipponbare cultivar (“OsHIPM-N”) (SEQ ID NO: 5). The DNA sequences were compared using ClustalW software. The start codon of OsHIPM-N is underlined, and the asterisks indicate matched bases.

FIGS. 5A-C are images showing protein interaction in yeast. Both bait in the pGilda vector and prey in the pB42AD vector carrying genes encoding the indicated proteins were transformed into yeast strain EGY48. Yeast transformants were screened in SD/gal-HT medium with leucine (“+Leu”) or without leucine (“−Leu”). 10 μl of 10-fold dilutions of yeast cell suspensions (OD₆₀₀=0.2) were plated and incubated for 5 days prior to photographing. Positive interaction resulted in yeast growth whether or not leucine was present. FIG. 5A shows the interaction of HrpN with HIPM and AtHIPM. FIG. 5B shows the self-interaction of HrpN, HIPM, and AtHIPM. FIG. 5C shows the interaction of HrpW with HIPM and AtHIPM.

FIG. 6 is a schematic diagram showing the map of an HIPM silencing construct, pART27-Hs-Has. The sense (“H-s”) and anti-sense (“H-as”) of the HIPM gene were cloned into the right and left sites of the intron in the pHANNIBAL vector, respectively. The NotI (“N”) DNA fragment indicated in the figure was transferred into a pART27 vector, and named pART27-Hs-Has. Also identified are the 35S promoter (“p35S”), the octopine synthase terminator (“Tocs”), the Nos promoter (“pNos”), the kanamycin resistance gene (“NPTII”), the Nos terminator (“Tnos”), the right border (“RB”), and the left border (“LB”).

FIG. 7 is a schematic diagram showing the map of an AtHIPM silencing construct, pART27-AtHs-AtHas. The sense (“AtH-s”) and anti-sense (“AtH-as”) of the HIPM gene were cloned into the right and left sites of the intron in the pHANNIBAL vector, respectively. The NotI (“N”) DNA fragment indicated in the figure was transferred into a pART27 vector, and named pART27-AtHs-AtHas. Also identified are the 35S promoter (“p35S”), the octopine synthase terminator (“Tocs”), the Nos promoter (“pNos”), the kanamycin resistance gene (“NPTII”), the Nos terminator (“Tnos”), the right border (“RB”), and the left border (“LB”).

FIG. 8 is a schematic diagram illustrating two OsHIPM silencing constructs in a pANDA vector. The 430-bp and 340-bp fragments of the OsHIPM gene (“OsH”) from the Jefferson cultivar were cloned into a pENTR vector. These fragments were transferred separately into the pANDA vector by LR clonase reaction, and named pANDA-OsH1 and pANDA-OsH2. Also identified are the GUS linker, the anti-sense OsHIPM fragment (“OsH-as”), the sense OsHIPM fragment (“OsH-s”), the maize ubiquitin1 gene promoter (“pUbq”), the Nos terminator (“Tnos”), the kanamycin resistance gene (“NPTII”), the hygromycin resistance gene (“HPT”), the right border (“RB”), and the left border (“LB”).

FIG. 9 is a schematic diagram showing the map of an AtHIPM overexpression construct, pBI121-AtH. The full length AtHIPM gene was cloned into a pBI121 vector under control of the 35S promoter (“p35S”) and the Nos terminator (“Tnos”), and named pBI121-AtH. Also identified are the Nos promoter (“pNos”), the kanamycin resistance gene (“NPTII”), the right border (“RB”), and the left border (“LB”).

FIG. 10 is a schematic diagram showing the map of an OsHIPM overexpression construct, pCAMBIA1300-OsH. The full-length OsHIPM gene is shown cloned into a pCAMBIA1300 vector under control of the rice Actin1 promoter (“pActin1”) and the Pin2 terminator (“tPin2”). Also identified are the 35S promoter (“p35S”), the hygromycin phosphotransferase gene (“HPT”), the right border (“RB”), and the left border (“LB”).

FIG. 11 is an alignment of HIPM (SEQ ID NO: 2) and its orthologs AtHIPM (SEQ ID NO: 4) and OsHIPM-N (SEQ ID NO: 6) by ClustalW software.

FIG. 12 is a western blot showing that HrpN interacts with both HIPM and AtHIPM in vitro. HIPM and AtHIPM genes were cloned into the pFLAG-CTC vector with FLAG tag, and the hrpN gene was cloned into the pET24a vector with T7 tag. Both HIPM-FLAG and AtHIPM-FLAG and HrpN were overexpressed in Escherichia coli BL21 (DE3) for use in an in vitro pull-down assay. HrpN and HIPM-FLAG and AtHIPM-FLAG were detected with HrpN antibody (“α-HrpN”) and FLAG tag M2 antibody (“α-FLAG”), respectively. +, presence; −, absence.

FIGS. 13A-B show that the 198-aa N-terminal region of HrpN is required for interaction with HIPM. FIG. 13A illustrates serial deletions of the hrpN gene generated in the pGilda vector. Expression of each deletion clone was checked in yeast using the LexA antibody (+, expressed; −, not expressed). Interaction with HIPM was determined in yeast with pB42AD-HIPM (++, strong interaction; ±, very weak interaction; −, no interaction). FIG. 13B illustrates the defense and growth domains of HrpN as characterized by personnel of Eden Bioscience Corporation.

FIG. 14 is a schematic diagram of the significant domains found in HIPM and its orthologs. The C-termini (“C”), N-termini (“N”), signal peptides (“SP”), and transmembrane domains (“TM”) are indicated.

FIGS. 15A-E are images showing that both HIPM and AtHIPM have functional signal peptides. The full-length cDNAs of HIPM (“HIPM¹⁻⁶⁰”) (FIG. 15A) and AtHIPM (“AtHIPM¹⁻⁵⁹”) (FIG. 15B) and their truncated forms (“HIPM²¹⁻⁶⁰” (FIG. 15C) and “AtHIPM²¹⁻⁵⁹” (FIG. 15D)), in which DNA encoding the first 20 amino acids were deleted, were cloned, in frame, with the suc2 gene in the pYSST0 vector. These constructs were transformed into yeast strain DBYα2445. Growth of the transformants was determined in a sucrose medium, and compared to growth of yeast transformed with the vector alone (FIG. 15E). As shown in FIGS. 15A-B, full-length HIPM and AtHIPM resulted in growth of the yeast transformants on sucrose medium. As shown in FIGS. 15C-D, the deletion clones did not confer growth.

FIGS. 16A-F are confocal micrographs showing that both HIPM and AtHIPM associate, in clusters, with plasma membranes of plant cells. Full-length HIPM and AtHIPM were cloned, in frame, with smGFP in the vector pCAMBIA2300. The GFP fusion proteins were transiently expressed in N. benthamiana by agroinfiltration. GFP signal was determined in mesophyll cells of leaves using confocal microscopy. In the untransformed cells (FIG. 16A), some green auto-fluorescence was detected only in the apoplasts but not inside plant cells, while in the GFP-transformed cells (FIG. 16B), green fluorescence existed throughout the cytoplasm. In the cells transformed with HIPM-GFP (FIG. 16C and FIG. 16E (top)) or AtHIPM-GFP (FIG. 16D), green fluorescence localized only to plasma membranes in water. In a 0.8 M mannitol solution (FIG. 16F (bottom)), cell shapes were irregular due to plasmolysis, unlike the turgid round shapes observed in water (FIG. 16E (bottom)). Green fluorescence was coincident with plasma membranes (FIG. 16F (top)).

FIGS. 17A-C are RT-PCR gels showing that both HIPM and AtHIPM are expressed more strongly in flowers than in leaves of apple and in stems of Arabidopsis. 0.7 μg of total RNA was used for RT-PCR, and EF-1α and genomic DNAs were used as internal controls. FIG. 17A shows HIPM expression in tissues of apple. Total RNA isolated from shoot (“S”), leaf (“L”), and four stages of flowers (tight cluster (“TC”), pink (“P”), full bloom (“F”) and 6 days after full bloom (“6F”)) was used. HIPM transcripts (250-bp) were detected using the primers indicated. FIG. 17B shows HIPM expression in leaves following inoculation with E. amylovora. Total RNA isolated from leaves at 6, 12, 22, and 45 hours after inoculation was used, and 250-bp HIPM transcripts were detected using the primers indicated in FIG. 17A. FIG. 17C shows AtHIPM expression in different tissues of Arabidopsis. Total RNA isolated from rosette leaves (“RL”), inflorescent shoots (“IS”), closed flowers (“CF”), open flowers (“OF”), and siliques (“S”), was used; genomic DNA (“G”) was used as a control. 290-bp transcripts of AtHIPM were detected using the primers indicated in FIG. 17A.

FIGS. 18A-C show that AtHIPM is needed for Arabidopsis to exhibit enhanced growth in response to HrpN. FIG. 18A is a RT-PCR gel of AtHIPM expression in a mutant line. 0.7 μg of total RNA isolated from rosette leaves was used, 290-bp AtHIPM transcripts were detected using the primers shown in FIG. 17C, and EF-1α and genomic DNAs (“G”) were used as internal controls. FIG. 18B is a graph of the growth of roots in response to HrpN. Seeds (n=26) were treated with water (black bar) or 15 μg/ml of HrpN (white bar) for 6 hours, and placed in a line on 0.5×Murashige & Skoog (“MS”) medium plates, which were maintained vertically. Root length was measured 10 days after seed placement. Length was converted to percentage relative to that of wild-type plants treated with water as 100%. FIG. 18C is a graph of top growth in response to HrpN. 3-week-old plants (n=15) were sprayed with water (black bar) or 15 μg/ml of HrpN (white bar), and leaf length was measured one week after spraying. Length was converted to percentage relative to that of wild-type plants treated with water as 100%. (“Col,” the wild-type Columbia; “A2,” the T-DNA-inserted mutant line.)

FIGS. 19A-C show that overexpression of AtHIPM reduces root length in Arabidopsis. FIG. 19A is a RT-PCR gel of AtHIPM expression in AtHIPM overexpressing Arabidopsis plants. 0.7 μg of total RNA isolated from rosette leaves was used, and 290-bp AtHIPM transcripts were detected using the primers shown in FIG. 17C. EF-1α and genomic DNAs (“G”) were used as internal controls. FIG. 19B is a graph of root growth of AtHIPM overexpressing Arabidopsis plants. Seeds (n=33) were placed in a line on 0.5×MS medium plates, which were maintained vertically. Root length was measured 10 days after seed placement. Length was converted to percentage relative to that of wild-type plants as 100%. FIG. 19C is a graph of top growth of AtHIPM overexpressing Arabidopsis plants. Leaf length of 3-week-old plants (n=16) grown in pots was measured. Length was converted to percentage relative to that of wild-type plants as 100%.

### DETAILED DESCRIPTION OF THE INVENTION

The present invention relates to a nucleic acid molecule configured to increase or decrease expression of a nucleic acid molecule that encodes a HrpN-interacting protein. The HrpN-interacting protein is (i) a protein having an amino acid sequence selected from the group of SEQ ID NO: 2, SEQ ID NO: 4, and SEQ ID NO: 6; (ii) a protein encoded by a nucleotide sequence of SEQ ID NO: 1, SEQ ID NO: 3, and SEQ ID NO: 5; and (iii) a protein at least 90% homologous and/or identical to the protein of (i) or (ii).

HrpN (harpin) of Erwinia amylovora, the first cell-free elicitor of the hypersensitive response, plays a critical role in the virulence of the fire blight pathogen. Moreover, HrpN promotes growth and induces systemic acquired resistance (“SAR”) after plants are treated with the protein. To determine the bases of the effects of HrpN, a HrpN-interacting protein(s) in apple, a host, were sought using a yeast two-hybrid assay. A single positive clone, designated HIPM (HrpN-interacting protein from Malus), was found. HIPM, a 6.5-kDa protein, interacts with HrpN in vitro. Deletion analysis showed that the 198-aa N-terminal region of HrpN is required for interaction with HIPM. HIPM orthologs were found in Arabidopsis thaliana (AtHIPM) and rice (OsHIPM). HrpN also interacted with AtHIPM in yeast and in vitro, and both HIPM and AtHIPM interacted with HrpW, the second harpin of E. amylovora. Domain analyses of HIPM and AtHIPM showed that they have functional signal peptides and they associate, in clusters, with plasma membranes. Domain analysis of OsHIPM-N using its amino acid sequence showed that it has a putative signal peptide and a putative transmembrane domain like HIPM, indicating that OsHIPM-N functions similarly to HIPM and AtHIPM. Both HIPM and AtHIPM are expressed constitutively. However, they are more strongly expressed in apple and Arabidopsis flowers than in leaves and stems. Arabidopsis with a loss-of-function mutation in AtHIPM are larger than parent plants, and they did not exhibit enhanced plant growth in response to treatment with HrpN. Overexpression of AtHIPM consistently resulted in smaller plants. These results indicate that HIPM, AtHIPM, and OsHIPM-N function as a negative regulators of plant growth and mediate enhanced growth that results from treatment with HrpN.

The HrpN-interacting protein from apple (“HIPM”) has an amino acid sequence of SEQ ID NO: 2, as follows:

Met Gly Gly Arg Gly Val Ile Gly Asp Arg Trp Ser
Met Arg Ile Leu Trp Ala Cys Ala Ile Gly Ser Ala
Val Ser Leu Tyr Met Val Ala Val Asp Arg Gln Leu
Lys Asn Arg Glu Arg Ala Leu Ala Glu Glu Leu Lys
Ala Met Glu Ala Glu Ser Gly Ser Gly Glu Ile Val.

HIPM is encoded by a nucleic acid molecule which has a nucleotide sequence of SEQ ID NO: 1 as follows:

AATTCCCCCGTTTCTCTCTTTCCCTCTCGCCGCTCTCTCCTCCATCTCCG
TCCCCAAACGCTAGGTGTGTCTCCGCCAAACCACTAGATATGGGAGGC
AGAGGAGTTATCGGGGATCGATGGTCCATGAGGATTCTCTGGGCGTGT
GCAATTGGAAGTGCTGTCAGCCTATATATGGTTGCTGTGGACAGACAA
CTAAAGAACAGGGAACGAGCGCTGGCCGAAGAGTTAAAGGCCATGGA
GGCAGAATCAGGCAGCGGTGAGATTGTTTGACTGTTGATAAATTATAG
GCAATAACTAGCTTAGAGCTTTCTAGATTTCCCAAGTTCGCATCTGTCA
TTTTGGCCATTGTGAGGATTATGTAAAATGTTGTTTGTTGTTCCCATTC
GGAATCAAGTTTTAGGGGGTTTACCCCGCTGACAAG.

The HrpN-interacting protein from Arabidopsis thaliana (“AtHIPM”) has an amino acid sequence of SEQ ID NO: 4, as follows:

Met Gly Ser Arg Gly Ile Ile Asn Asp Lys Trp Ser
Met Arg Ile Leu Trp Gly Cys Ala Ile Gly Ser Ala
Ile Gly Leu Tyr Met Val Ala Val Glu Arg Gln Thr
Gln Asn Arg Ala Arg Ala Met Ala Glu Ser Leu Arg
Ala Ala Glu Ser Gln Gly Asp Gly Asp Asn Val.

AtHIPM is encoded by a nucleic acid molecule which has a nucleotide sequence of SEQ ID NO: 3 as follows:

AATTGTTTTAAAATTACAAATTAGTCCGTTCTTTTATTCCCGTACTCGTT
CCTTCTTCTTCTTCTTCCTCTCATCGTCATTTTCTCGATTCTCACTCTTC
CGGTCACCGACTAATTCTGAATAAGGTTTATCAAAAGAATAAGAATAAGT
GGATAAAAAGCTAGCTTTGAAAGAGTTATTGCAGAGAAAAAAAATGGGAT
CGAGAGGGATTATCAACGATAAGTGGTCAATGAGGATTCTATGGGGTTGT
GCTATCGGAAGTGCTATTGGTTTATACATGGTTGCTGTAGAGAGACAAAC
TCAGAACAGGGCTCGTGCTATGGCTGAGAGTTTGAGAGCTGCTGAATCAC
AAGGTGATGGTGATAATGTCTAATATCTACCAAGTAGTGCTCAGTTGAAT
ACTCTCAGTTGAGTTTTTTTTTTTGGTGTTTGTTTTTGTTATAATGACTT
CTTCTGCCAAGATGGTGTTGATGTAGTTTCTTTTTTGCAAATAATCGTAA
TAAGGTTTCGAAACTTGGAGAGTTGAAGTTGCTGAACATACGATTTGTGT
TATCGCAAAAAAAGTTATTTCTTATGCCTG.

The HrpN-interacting protein from the Nipponbare cultivar of rice (“OsHIPM-N”) has an amino acid sequence of SEQ ID NO: 6, as follows:

Met Gly Leu Gly Gly Arg Gly Val Val Gly Glu Arg
Trp Ser Gln Arg Val Leu Trp Leu Cys Ala Ile Gly
Ser Ala Val Ser Leu Tyr Tyr Val Ala Val Glu Arg
Gln Ala Gln Asn Arg Ala Arg Ala Val Ala Glu Gly
Leu Lys Ala Leu Asp Gly Ala Gly Ala Gly Glu Asp
Val.

This protein is encoded by a nucleic acid molecule which has a nucleotide sequence of SEQ ID NO: 5 as follows:

ACTCGGAGGCTGCGGCCCGCACGGCGAACGGAGCGGCGGCGCAGCTC
GCGCGATCAATCGTCGGCGGCAGCGGCGGCGGCGGCGGCGGGATGGG
GCTCGGGGGGCGAGGCGTGGTGGGGGAGAGGTGGTCGCAGCGCGTCC
TCTGGCTCTGCGCCATCGGCAGCGCCGTGAGCCTGTACTACGTGGCGG
TGGAGAGGCAGGCGCAGAACCGCGCGCGGGCGGTGGCCGAGGGGCTC
AAGGCCCTCGACGGCGCCGGCGCCGGAGAGGACGTGTGACTTCGCTGT
GTGCTGGAGAGGTGATCCCGGCCTGTGTAGAGACGGCCTCTCTGTTCG
AGCTCGAAACGAGTTATATTTTTGCTTACCTTGTTTCTTGTTTCATGAA
ATTTTCGCAATAATAATGTACTAGTAATTGCCCCCTTTGTCATTGCGAT
AACTGGATTACAATTTGCGATATGGGAGCCAGAAATGATGGCCGAAAT
GAATGTCATCTGTTTGTTCT.

The HrpN-interacting protein from the Jefferson cultivar of rice is encoded by a nucleic acid molecule which has a nucleotide sequence of SEQ ID NO: 7 as follows:

ACTCGGAGGCTGCGGCCCGCACGGCGAACGGAGCGGCGGCGCAGCTC
GCGCGATCAATCGCCGCCTAATCGCAGTGGTTTGATCCGGCGGCGTCG
CCGCGTGTGCAGGCCTGTACTACGTGGCGGTGGAGAGGCAGGCGCAG
AACCGCGCGCGGGCGGTGGCCGAGGGGCTCAAGGCCCTCGACGGCGC
CGGCGCCGGAGAGGACGTGTGACTTCGCTGTGTGCTGGAGAGGTGATC
CCGGCCTGTGTAGAGACGGCCTCTCTGTTCGAGCTCGAAACGAGTTAT
ATTTTTGCTTACCTTGTTTCTTGTTTCATGAAATTTTCGCAATAATAATG
TACTAGTAATTGCCCCCTTTGTCATTGCGATAACTGGATTACAATTTGC
GATATGGGAGCCAGAAATGATGGCCGAAATGAATGTCATCTGTTTG.

Suitable nucleic acid molecules of the present invention include those configured to increase or decrease expression of a nucleic acid molecule that encodes a HrpN-interacting protein. For example, nucleic acid molecules that include the nucleotide sequence of SEQ ID NO: 1 and/or SEQ ID NO: 31 may be used to increase expression of HIPM; a nucleic acid molecule that includes the nucleotide sequence of SEQ ID NO: 3 and/or SEQ ID NO: 32 may be used to increase expression of AtHIPM; and nucleic acid molecules that include the nucleotide sequence of SEQ ID NO: 5 and/or SEQ ID NO: 33 may be used to increase expression of OsHIPM. Nucleic acid molecules that include the nucleotide sequence of SEQ ID NO: 27 or suitable fragments thereof may be used to decrease expression of HIPM; nucleic acid molecules that include the nucleotide sequence of SEQ ID NO: 28 or suitable fragments thereof may be used to decrease expression of AtHIPM; and nucleic acid molecules that include the nucleotide sequence of SEQ ID NO: 29 or SEQ ID NO: 30, or suitable fragments thereof, may be used to decrease expression of OsHIPM. In this context, suitable fragments include any fragment of sufficient length to effect silencing of expression of an endogenous HrpN-interacting protein. Generally, fragments of at least around 20 nucleotides in length are sufficient. One of ordinary skill in the art will recognize that other suitable nucleic acid molecules, including those that may be used to increase or decrease expression of nucleic acid molecules that encode a protein at least 90% homologous and/or identical to the proteins of (i) or (ii) set forth above, may also be designed considering the nucleotide sequence of the nucleic acid molecule whose expression is to be increased/decreased, and/or the amino acid sequence of the HrpN-interacting protein it encodes.

Suitable nucleic acid molecules include those that interfere with or inhibit expression of the nucleic acid molecule that encodes the Hrpn-interacting protein by RNA interference (“RNAi”). These include, but are not limited to, nucleic acid molecules including RNA molecules which are homologous to the target gene or genomic sequence or a fragment thereof, short interfering RNA (“siRNA”), short hairpin or small hairpin RNA (“shRNA”), and small molecules which interfere with or inhibit expression of a target gene by RNAi.

Preferably, the nucleic acid molecule of the present invention configured to decrease expression of the nucleic acid molecule encoding the HrpN-interacting protein is siRNA. In one embodiment, the siRNA is an siRNA targeting HIPM, AtHIPM, and/or OsHIPM. Preferred siRNAs include those described in Example 12 (see Table 1). Other siRNA nucleic acid molecules of the present invention may be readily designed and tested, as will be apparent to one of ordinary skill in the art.

RNAi is an evolutionarily-conserved process whereby the expression or introduction of RNA of a sequence that is identical or highly similar to a target gene results in the sequence-specific degradation or specific post-transcriptional gene silencing of mRNA transcribed from that targeted gene (see Coburn & Cullen, “Potent and Specific Inhibition of Human Immunodeficiency Virus Type 1 Replication by RNA Interference,” J. Virol. 76(18):9225-31 (2002), which is hereby incorporated by reference in its entirety), thereby inhibiting expression of the target gene. In one embodiment, the RNA is double stranded RNA (“dsRNA”). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “decrease expression” includes any decrease in expression or protein activity or level of the target gene or protein encoded by the target gene as compared to a situation wherein no RNA interference has been induced. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

siRNA, also referred to herein as “small interfering RNA” is defined as an agent which functions to inhibit expression of a target gene, e.g., by RNAi. An siRNA may be chemically synthesized, produced by in vitro transcription, or produced within a host cell. The siRNA molecules can be single-stranded or double stranded.

siRNAs also include small hairpin (also called stem loop) RNAs (“shRNAs”). In one embodiment, these shRNAs are composed of a short (e.g., about 19 to about 25 nucleotide) antisense strand followed by a nucleotide loop of about 5 to about 9 nucleotides and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter or another promoter (see, e.g., Stewart et al., “Lentivirus-delivered Stable Gene Silencing by RNAi in Primary Cells,” RNA 9(4):493-501 (2003), which is hereby incorporated by reference in its entirety).

Preferably, the siRNA molecules have a length of about 15 to about 40 nucleotides in length, (preferably about 15 to about 28 nucleotides, more preferably about 19 to about 25 nucleotides in length, and most preferably about 19, 20, 21, 22, or 23 nucleotides in length).

Such molecules can be blunt ended or comprise overhanging ends, e.g., a 3′ and/or 5′ overhang. Preferably, the overhangs have a length of about 0 to about 6 nucleotides, about 1 to about 3 nucleotides, or about 2 to about 4 nucleotides. The existence/length of the overhang is independent between the two strands, i.e., the length of the overhang on one strand is not dependent on the length of the overhang on the second strand, and one strand could be can be blunt-ended while the other has an overhang. The 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium. In a particular embodiment, the RNA of the present invention comprises about 19, 20, 21, or 22 nucleotides which are paired and which have overhangs of from about 1 to about 3, particularly about 2, nucleotides on both 3′ ends of the RNA.

The siRNA molecules of the present invention can also comprise a 3′ hydroxyl group. Preferably the siRNA is capable of promoting RNA interference through degradation or specific post-transcriptional gene silencing of the target mRNA.

siRNA molecules need not be limited to those molecules containing only RNA, but, for example, further encompasses chemically modified nucleotides and non-nucleotides, and also include molecules in which a ribose sugar molecule has been substituted for another sugar molecule or a molecule which performs a similar function. Moreover, a non-natural linkage (e.g., a phosphorothioate linkage) between nucleotide residues may be used. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′-O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful decrease in expression of the target nucleic acid can also be incorporated.

The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., “RNA Interference in Mammalian Cells by Chemically-modified RNA,” Biochem. 42:7967-75 (2003), which is hereby incorporated by reference in its entirety. Most of the useful modifications to the siRNA molecules can be introduced using chemistries established for antisense oligonucleotide technology.

An siRNA may be substantially homologous to the target nucleic acid molecule or to a fragment thereof As used herein, the term “homologous” is defined as being substantially identical, sufficiently complementary, or similar to the target mRNA (or a fragment thereof) to effect RNA interference of the target. In addition to native RNA molecules, RNA suitable for inhibiting or interfering with the expression of a target sequence include RNA derivatives and analogs. Preferably, the siRNA is identical to its target allele so as to prevent its interaction with the normal allele.

The nucleic acid molecules of the present invention can be obtained using a number of techniques known to those of skill in the art. For example, nucleic acid molecules can be chemically synthesized or recombinantly produced using methods known in the art, such as using appropriately protected ribonucleoside phosphoramidites and a conventional DNA/RNA synthesizer (see, e.g., Elbashir et al., “Duplexes of 21-Nucleotide RNAs Mediate RNA Interference in Cultured Mammalian Cells,” Nature 411:494-8 (2001); Elbashir et al., “RNA Interference Is Mediated by 21- and 22-Nucleotide RNAs,” Genes Devel. 15:188-200 (2001); Harborth et al., “Identification of Essential Genes in Cultured Mammalian Cells Using Small Interfering RNAs,” J. Cell Science 114:4557-65 (2001); Masters et al., “Short Tandem Repeat Profiling Provides an International Reference Standard for Human Cell Lines,” Proc. Nat'l Acad. Sci. USA 98:8012-7 (2001); Tuschl et al., “Targeted mRNA Degradation by Double-stranded RNA In Vitro,” Genes Devel. 13:3191-7 (1999), which are hereby incorporated by reference in their entirety). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, nucleic acid molecules, e.g., siRNA molecules, are not overly difficult to synthesize and are readily provided in a quality suitable for their use. In addition, dsRNAs and other double-stranded nucleic acid molecules can be expressed as stem loop structures encoded by plasmid vectors, retroviruses, and lentiviruses (Paddison et al., “Short Hairpin RNAs (shRNAs) Induce Sequence-specific Silencing in Mammalian Cells,” Genes Dev. 16:948-58 (2002); McManus et al., “Gene Silencing Using Micro-RNA Designed Hairpins,” RNA 8:842-50 (2002); Paul et al., “Effective Expression of Small Interfering RNA in Human Cells,” Nat. Biotechnol. 20:505-8 (2002); Miyagishi & Taira, “U6 Promoter-driven siRNAs With Four Uridine 3′ Overhangs Efficiently Suppress Targeted Gene Expression in Mammalian Cells,” Nat. Biotechnol. 20:497-500 (2002); Sui et al., “A DNA Vector-based RNAi Technology to Suppress Gene Expression in Mammalian Cells,” Proc. Nat'l Acad. Sci. USA 99:5515-20 (2002); Brummelkamp et al., “Stable Suppression of Tumorigenicity by Virus-mediated RNA Interference,” Cancer Cell 2:243-7 (2002); Lee et al., “Expression of Small Interfering RNAs Targeted Against HIV-1 Rev Transcripts in Human Cells,” Nat. Biotechnol. 20:500-5 (2002); Yu et al., “RNA Interference by Expression of Short-interfering RNAs and Hairpin RNAs in Mammalian Cells,” Proc. Nat'l Acad. Sci. USA 99:6047-52 (2002); Zeng et al., “Both Natural and Designed Micro RNAs Can Inhibit the Expression of Cognate mRNAs When Expressed in Human Cells,” Mol. Cell 9:1327-33 (2002); Rubinson et al., “A Lentivirus-based System to Functionally Silence Genes in Primary Mammalian Cells, Stem Cells and Transgenic Mice by RNA Interference,” Nat. Genet. 33:401-6 (2003); Stewart et al., “Lentivirus-delivered Stable Gene Silencing by RNAi in Primary Cells,” RNA 9:493-501 (2003); Miki & Shimamoto, “Simple RNAi Vectors for Stable and Transient Suppression of Gene Function in Rice,” Plant Cell Physiol. 45(4):490-5 (2004); Wesley et al., “Construct Design for Efficient, Effective and High-throughput Gene Silencing in Plants,” Plant J. 27:581-90 (2001), which are hereby incorporated by reference in their entirety). These vectors generally have a polIII promoter upstream of the nucleic acid molecule (e.g., dsRNA) and can express sense and antisense RNA strands separately and/or as a hairpin structures. Within cells, Dicer processes the short hairpin RNA into effective siRNA.

The targeted region of the siRNA molecule of the present invention can be selected from a given target gene sequence, e.g., SEQ ID NO: 1, SEQ ID NO: 3, or SEQ ID NO: 5, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N₁₉)TT (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search may be extended using the motif NA(N₂₁), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (“siRNPs”) are formed with approximately equal ratios of sense and antisense target RNA-cleaving siRNPs (Elbashir et al., “Duplexes of 21-Nucleotide RNAs Mediate RNA Interference in Cultured Mammalian Cells,” Nature 411:494-8 (2001); Elbashir et al., “RNA Interference Is Mediated by 21- and 22-Nucleotide RNAs,” Genes Devel. 15:188-200 (2001), which are hereby incorporated by reference in their entirety).

The siRNA preferably targets only one sequence. Each of the nucleic acid molecules configured to decrease expression of the HrpN-interacting protein-encoding nucleic acid molecule, such as siRNAs, can be screened for potential off-target effects using, for example, expression profiling. Such methods are known to one skilled in the art and are described, for example, in Jackson et al., “Expression Profiling Reveals Off-target Gene Regulation by RNAi,” Nat. Biotechnol. 6:635-7 (2003), which is hereby incorporated by reference in its entirety. In addition to expression profiling, one may also screen the potential target sequences for similar sequences in the sequence databases, including but not limited to NCBI, BLAST, Derwent, and GenSeq, as well as commercially available oligosynthesis companies such as Oligoengine®, to identify potential sequences which may have off-target effects. For example, according to Jackson et al., “Expression Profiling Reveals Off-target Gene Regulation by RNAi,” Nat. Biotechnol. 6:635-7 (2003), which is hereby incorporated by reference in its entirety, 15 or perhaps as few as 11 contiguous nucleotides of sequence identity are sufficient to direct silencing of non-targeted transcripts. Therefore, one may initially screen the proposed siRNAs to avoid potential off-target silencing using the sequence identity analysis by any known sequence comparison method, such as BLAST.

The siRNAs of the present invention are preferably designed so as to maximize the uptake of the antisense (guide) strand of the siRNA into RNA-induced silencing complex (“RISC”) and thereby maximize the ability of RISC to target HrpN-interacting protein mRNA for degradation. This can be accomplished by looking for sequences that have the lowest free energy of binding at the 5′-terminus of the antisense strand. The lower free energy would lead to an enhancement of the unwinding of the 5′ end of the antisense strand of the siRNA duplex, thereby ensuring that the antisense strand will be taken up by RISC and direct the sequence-specific cleavage of the HrpN-interacting protein mRNA.

The nucleic acid molecules of the present invention may be introduced into nucleic acid constructs, plants, or plant cells along with components that perform one or more of the following activities: enhance uptake of the nucleic acid molecule, inhibit annealing of single strands and/or stabilize single strands, or otherwise facilitate delivery to the target and increase the ability of the nucleic acid molecule to increase or decrease expression of the HrpN-interacting protein-encoding nucleic acid molecule. It will be apparent to one of skill in the art that RNA may be introduced into a target by introducing its corresponding DNA molecule into the target such that the DNA molecule is transcribed, resulting in production of the RNA molecule.

One or more nucleic acid molecules of the present invention may be incorpated into a nucleic acid construct. The nucleic acid construct according to this aspect of the present invention includes a nucleic acid molecule of the present invention, a 5′ promoter sequence, and a 3′ terminator sequence, operatively coupled to permit transcription of the nucleic acid molecule. The nucleic acid molecule can be in sense orientation or antisense orientation.

In a preferred embodiment, the nucleic acid molecule is configured to increase expression of the HrpN-interacting protein. In a preferred embodiment, the nucleic acid molecule of the present invention encodes the HrpN-interacting protein and is in sense orientation. Suitable nucleic acid molecules according to this embodiment include, without limitation, nucleic acid molecules that include the nucleotide sequence of SEQ ID NO: 1, SEQ ID NO: 3, SEQ ID NO: 5, SEQ ID NO: 31, SEQ ID NO: 32, or SEQ ID NO: 33.

In another preferred embodiment, the nucleic acid molecule is configured to decrease expression of the HrpN-interacting protein. In this embodiment the nucleic acid molecule may, e.g., be positioned in the nucleic acid construct to result in suppression or interference of endogenous mRNA encoding the HrpN-interacting protein; be an antisense form of at least a portion of a HrpN-interacting protein-encoding nucleic acid molecule; or include a first segment encoding at least a portion of a HrpN-interacting protein, a second segment in an antisense form of the first segment, and a third segment linking the first and second segments. Exemplary nucleic acid molecules according to this embodiment include, without limitation, those that include a nucleotide sequence of SEQ ID NO: 27, SEQ ID NO: 28, SEQ ID NO: 29, SEQ ID NO: 30, or suitable fragments of these sequences.

The nucleic acid molecules and constructs of the present invention can be incorporated in cells using conventional recombinant technology. Generally, this involves inserting the nucleic acid molecule or construct into an expression system to which the nucleic acid molecule is heterologous (i.e., not normally present). The heterologous nucleic acid molecule/construct is inserted into the expression system or vector in proper sense orientation and correct reading frame. The vector contains the necessary elements for the transcription of the inserted sequences. It may also contain the necessary elements for the translation of the inserted sequences, e.g., when overexpression of the Hrpn-interacting protein is desired. Thus, the present invention also relates to an expression vector that includes the nucleic acid construct of the present invention.

U.S. Pat. No. 4,237,224 to Cohen & Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture.

Recombinant genes may also be introduced into viruses, such as vaccina virus. Recombinant viruses can be generated by transfection of plasmids into cells infected with virus.

Suitable vectors include, but are not limited to, the viral vectors such as lambda vector system gt11, gt WES.tB, Charon 4; and plasmid vectors such as pBR322, pBR325, pACYC177, pACYC1084, pUC8, pUC9, pUC18, pUC19, pLG339, pR290, pKC37, pKC11, SV 40, pBluescript II SK+/− or KS+/− (see STRATAGENE, STRATAGENE CLONING SYSTEMS (1993), which is hereby incorporated by reference in its entirety), pQE, pIH821, pGEX, pET series (see Studier et. al., “Use of T7 RNA Polymerase to Direct Expression of Cloned Genes,” Methods Enzymol. 185:60-89 (1990), which is hereby incorporated by reference in its entirety), and any derivatives thereof. Preferred vectors for plant transformation include, for example, pBI121, pART27, pCAMBIAs, pBTEX, and pGPTV-Bar. Recombinant molecules can be introduced into cells via transformation, particularly transduction, conjugation, mobilization, or electroporation. The nucleic acid molecules/constructs are cloned into the vector using standard cloning procedures in the art, as described by SAMBROOK & RUSSELL, MOLECULAR CLONING (1989), which is hereby incorporated by reference in its entirety.

A variety of host-vector systems may be utilized to express the nucleic acid molecule. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and mRNA translation).

Transcription of DNA is dependent upon the presence of a promotor which is a DNA sequence that directs the binding of RNA polymerase and thereby promotes mRNA synthesis. The DNA sequences of eucaryotic promotors differ from those of procaryotic promotors. Furthermore, eucaryotic promotors and accompanying genetic signals may not be recognized in or may not function in a procaryotic system, and, further, procaryotic promoters are not recognized and do not function in eucaryotic cells.

Similarly, translation of mRNA in procaryotes depends upon the presence of the proper procaryotic signals which differ from those of eucaryotes. Efficient translation of mRNA in procaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA and probably promote binding of mRNA to ribosomes by duplexing with the rRNA to allow correct positioning of the ribosome. For a review on maximizing gene expression, see Roberts & Lauer, “Maximizing Gene Expression on a Plasmid Using Recombination In Vitro,” Methods Enzymol. 68:473-82 (1979), which is hereby incorporated by reference in its entirety.

Promotors vary in their “strength” (i.e., their ability to promote transcription). For the purposes of expressing a cloned gene, it is desirable to use strong promotors in order to obtain a high level of transcription and, hence, expression of the gene. Depending upon the host cell system utilized, any one of a number of suitable promotors may be used. For instance, when cloning in E. coli, its bacteriophages, or plasmids, promoters such as the T7 phage promoter, lac promotor, trp promotor, recA promotor, ribosomal RNA promotor, the P_{R} and P_{L} promotors of coliphage lambda and others, including but not limited, to lacUV5, ompF, bla, 1 pp, and the like, may be used to direct high levels of transcription of adjacent DNA segments. Additionally, a hybrid trp-lacUV5 (tac) promotor or other E. coli promotors produced by recombinant DNA or other synthetic DNA techniques may be used to provide for transcription of the inserted gene. When the promoter is meant to be expressed in transgenic plants, suitable promoters include, e.g., constitutive promoters, inducible promoters, tissue specific promoters, and organ-specific promoters. Preferred constitutive and inducible promoters include 35S (constitutive), nos (constitutive), rice actin 1 (constitutive), and hsr203J (inducible). Preferred tissue specific plant promoters include rbcS (leaf specific) and catB (root specific). Preferred organ-specific plant promoters include RTS (anther-specific) and alpha amy3 (seed specific).

Bacterial host cell strains and expression vectors may be chosen which inhibit the action of the promotor unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in procaryotic cells. These transcription and translation initiation signals may vary in “strength” as measured by the quantity of gene specific messenger RNA and protein synthesized, respectively. The DNA expression vector, which contains a promotor, may also contain any combination of various “strong” transcription and/or translation initiation signals. For instance, efficient translation in E. coli requires an SD sequence about 79 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

The constructs of the present invention also include a terminator sequence. Suitable transcription termination sequences include the termination region of a 3′ non-translated region. This will cause the termination of transcription and the addition of polyadenylated ribonucleotides to the 3′ end of the transcribed mRNA sequence. The termination region or 3′ non-translated region will be additionally one of convenience. The termination region may be native with the promoter region or may be derived from another source, and preferably includes a terminator and a sequence coding for polyadenylation. Suitable 3′ non-translated regions include but are not limited to: (1) the 3′ transcribed, non-translated regions containing the polyadenylated signal of Agrobacterium tumor-inducing (“Ti”) plasmid genes, such as the nopaline synthase (“NOS”) gene or the 35S promoter terminator gene; and (2) plant genes like the soybean 7S storage protein genes and the pea small subunit of the ribulose 1,5-bisphosphate carboxylase-oxygenase (“ssRUBISCO”) E9 gene.

Once the nucleic acid molecule/construct of the present invention has been cloned into an expression system, it may be incorporated into a host cell. Such incorporation can be carried out by the various forms of transformation noted above, depending upon the vector/host cell system. Suitable host cells include, but are not limited to, bacteria, virus, yeast, mammalian, insect, plant, and the like. Preferably, the host cell is a bacterial cell or a plant cell. Suitable plant cells include cells of the plants identified herein.

The present invention also relates to host cells, transgenic plants, and transgenic plant seeds transformed with the nucleic acid constructs disclosed herein. In one embodiment, the host cell, plant, or plant seed is transformed with first and second of the nucleic acid constructs with the first nucleic acid construct encoding at least a portion of a HrpN-interacting protein in sense orientation and the second nucleic acid construct encoding at least a portion of a HrpN-interacting protein in antisense form.

In producing transgenic plants, the nucleic acid construct in a vector described above can be microinjected directly into plant cells by use of micropipettes to transfer mechanically the recombinant nucleic acid molecule (Crossway et al., “Integration of Foreign DNA Following Microinjection of Tobacco Mesophyll Protoplasts,” Mol. Gen. Genetics 202:179-85 (1986), which is hereby incorporated by reference in its entirety). The genetic material may also be transferred into the plant cell using polyethylene glycol (Krens et al., “In Vitro Transformation of Plant Protoplasts With Ti-plasmid DNA,” Nature 296(5852):72-4 (1982), which is hereby incorporated by reference in its entirety).

Another approach to transforming plant cells with the nucleic acid construct is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford et al., which are hereby incorporated by reference in their entirety. Generally, this procedure involves propelling inert or biologically active particles at the cells under conditions effective to penetrate the outer surface of the cell and to be incorporated within the interior thereof. When inert particles are utilized, a vector containing the nucleic acid construct can be introduced into the cell by coating the particles with the vector containing that heterologous nucleic acid construct. Alternatively, the target cell can be surrounded by the vector so that the vector is carried into the cell by the wake of the particle. Biologically active particles (e.g., dried bacterial cells containing the vector and the heterologous nucleic acid construct) can also be propelled into plant cells.

Yet another method of introduction is fusion of protoplasts with other entities, either minicells, cells, lysosomes, or other fusible lipid-surfaced bodies (Fraley et al., “Liposome-mediated Delivery of Tobacco Mosaic Virus RNA into Tobacco Protoplasts: A Sensitive Assay for Monitoring Liposome-protoplast Interactions,” Proc. Nat'l Acad. Sci. USA 79(6): 1859-63 (1982), which is hereby incorporated by reference in its entirety).

The nucleic acid molecule/construct may also be introduced into the plant cells by electroporation (Fromm et al., “Expression of Genes Transferred into Monocot and Dicot Plant Cells by Electroporation,” Proc. Nat'l Acad. Sci. USA 82:5824-8 (1985), which is hereby incorporated by reference in its entirety). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate.

Another method of introducing the nucleic acid molecule/construct into plant cells is to infect a plant cell with Agrobacterium tumefaciens or A. rhizogenes previously transformed with the nucleic acid molecule/construct. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C.

Agrobacterium is a representative genus of the gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue.

Heterologous genetic sequences can be introduced into appropriate plant cells by means of the Ti plasmid of A. tumefaciens or the Ri plasmid of A. rhizogenes. The Ti or Ri plasmid is transmitted to plant cells on infection by Agrobacterium and is stably integrated into the plant genome (St. Schell, “Transgenic Plants as Tools to Study the Molecular Organization of Plant Genes,” Science 237:1176-83 (1987), which is hereby incorporated by reference in its entirety).

Other suitable methods for transforming plant cells include vacuum infiltration and laser-beam transformation.

After transformation, the transformed plant cell may be regenerated.

Plant regeneration from cultured protoplasts is described in 1 HANDBOOK OF PLANT CELL CULTURES (David A. Evans et al. eds., 1st ed. 1983), which is hereby incorporated by reference in its entirety, I CELL CULTURE AND SOMATIC CELL GENETICS OF PLANTS (Indra K. Vasil ed., 1984), which is hereby incorporated by reference in its entirety, and III CELL CULTURE AND SOMATIC CELL GENETICS OF PLANTS (Indra K. Vasil ed., 1986), which is hereby incorporated by reference in its entirety.

It is known that practically all plants can be regenerated from cultured cells or tissues.

Means for regeneration vary between plant species, but generally a suspension of transformed protoplasts or a petri plate containing explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

After the expression cassette is stably incorporated in transgenic plants, it can be transferred to other plants by sexual crossing. Any of a number of standard breeding techniques can be used, depending upon the species to be crossed.

Once transgenic plants of this type are produced, the plants themselves can be cultivated in accordance with conventional procedure so that the nucleic acid molecule/construct is present in the resulting plants. Alternatively, transgenic seeds may be recovered from the transgenic plants. These seeds can then be planted in the soil and cultivated using conventional procedures to produce transgenic plants.

The nucleic acid molecules/constructs of the present invention can be utilized in conjunction with a wide variety of plants or their cells or seeds. Suitable plants include dicots and monocots. More particularly, useful crop plants include, e.g., alfalfa, apple, barley, bean, beet, broccoli, brussel sprout, cabbage, cauliflower, carrot, celery, chicory, citrus, corn, cotton, cucumber, eggplant, endive, garlic, grape, lettuce, maize, Malus, Medicago truncatula, melon, onion, parsnip, pea, peanut, pear, pepper, pine, pineapple, potato, pumpkin, radish, raspberry, rice, rye, soybean, sorghum, spinach, squash, strawberry, sugarcane, sunflower, sweet potato, tobacco, tomato, turnip, wheat, and zucchini. Examples of suitable ornamental plants are, e.g., Arabidopsis, carnation, chrysanthemum, crocus, daffodil, pelargonium, petunia, poinsettia, rose, Saintpaulia, Sandersonia aurantiaca, thaliana, and zinnia.

The present invention also relates to component parts and fruit of the transgenic plants of the present invention, and plant seeds produced from the these plants.

A further aspect of the present invention relates to a method of increasing or decreasing plant growth. This method involves providing a transgenic plant or plant seed transformed with a nucleic acid construct of the present invention and growing the transgenic plant or a transgenic plant grown from the transgenic plant seed under conditions effective to increase plant growth compared to non-transgenic plants.

The transgenic plant or plant seed may be provided as described herein.

With regard to the use of the nucleic acid molecules and contructs of the present invention to increase plant growth, various forms of plant growth enhancement or promotion can be achieved. This can occur as early as when plant growth begins from seeds or later in the life of a plant. For example, plant growth according to the present invention encompasses greater yield, increased quantity of seeds produced, increased percentage of seeds germinated, increased plant size, increased plant height, increased root growth, increased leaf growth, greater biomass, more and bigger fruit, earlier germination, earlier fruit and/or plant coloration, and earlier fruit and/or plant maturation. As a result, the present invention provides significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land. It is thus apparent that the present invention constitutes a significant advance in agricultural efficiency.

Without wishing to be bound by theory, such growth enhancement may result from decreased levels of HrpN-interacting protein in the plant.

Another aspect of the present invention relates to a method of imparting disease resistance to plants. This method involves providing a transgenic plant or plant seed transformed with a nucleic acid construct of the present invention and growing the transgenic plant or a transgenic plant grown from the transgenic plant seed under conditions effective to impart disease resistance to the plant compared to non-transgenic plants.

The transgenic plant or plant seed may be provided as described herein.

With regard to the use of the nucleic acid molecules and contructs of the present invention to impart disease resistance, various forms of disease resistance can be achieved. For example, absolute immunity against infection may not be conferred, but the severity of the disease may be reduced and symptom development delayed. Lesion number, lesion size, and extent of sporulation of fungal pathogens may be decreased. This method of imparting disease resistance has the potential for treating previously untreatable diseases, treating diseases systemically which might not be treated separately due to cost, and avoiding the use of infectious agents or environmentally harmful materials.

This aspect of the present invention is useful in imparting resistance to a wide variety of pathogens including viruses, bacteria, and fungi.

Without being bound by theory, this aspect of the present invention may be used to remove (or reduce) from a host plant, a protein (i.e., Hrp-interacting protein) that specifically interacts with a pathogen protein (i.e., harpin) that is needed to cause disease. Pathogens that produce harpins that have been shown to play a role in disease, and examples of such diseases, include, for example, Ralstonia solanacearum (Bacterial Wilt of tomato and potato), Pseudomonas syringae (Bacterial Speck of tomato and Halo Blight of bean), and Xanthomonas species (Bacterial Leaf Blight of rice caused by Xanthomonas oryzae; Bacterial spot of tomato and pepper caused by Xanthomonas vesicatoria).

Resistance to diseases mediated by, e.g., HrpN and/or HrpW can be imparted to plants in accordance with the present invention. Preferably, the disease according to this aspect of the present invention is fire blight.

The methods of the present invention can be utilized to treat a wide variety of plants or their seeds, as described above, to increase plant growth and/or impart disease resistance.

The present invention also relates to an isolated nucleic acid molecule comprising bases 90 to 269 of the nucleotide sequence of SEQ ID NO: 1.

A further aspect of the present invention relates to an isolated protein or polypeptide that includes the amino acid sequence of SEQ ID NO: 2.

Methods for producing proteins are well known in the art. For example, the gene encoding a protein of the present invention may be expressed in vitro or in vivo in bacterial cells, and the protein isolated. In another approach, chemical synthesis can be carried out using known amino acid sequences for the protein being produced.

Protein isolation procedures are well known, as described in Arlat et al., “PopA1, A Protein Which Induces a Hypersensitivity-like Response on Specific Petunia Genotypes, Is Secreted Via the Hrp Pathway of Pseudomonas solanacearum,” EMBO J. 13(3):543-53 (1994), which is hereby incorporated by reference in its entirety; He et al., “Pseudomonas syringae pv. syringae Harpin_{Pss}: A Protein That Is Secreted Via the Hrp Pathway and Elicits the Hypersensitive Response in Plants,” Cell 73:1255-66 (1993), which is hereby incorporated by reference in its entirety; and Wei et al., “Harpin, Elicitor of the Hypersensitive Response Produced by the Plant Pathogen Erwinia amylovora,” Science 257:85-8 (1992), which is hereby incorporated by reference in its entirety (see also U.S. Pat. No. 5,708,139 to Collmer et al.; U.S. Pat. No. 5,849,868 to Beer et al., which are hereby incorporated by reference in their entirety). Preferably, however, the protein or polypeptide of the present invention is produced recombinantly.

The protein or polypeptide of this aspect of the present invention is preferably produced in purified form (preferably at least about 80%, more preferably 90%, pure) by conventional techniques. Typically, the protein or polypeptide is secreted into the growth medium of recombinant host cells. Alternatively, the protein or polypeptide is produced but not secreted into growth medium. In such cases, to isolate the protein, the host cell (e.g., E. coli) carrying a recombinant plasmid is propagated, lysed by sonication, heat, differential pressure, or chemical treatment, and the homogenate is centrifuged to remove bacterial debris. The supernatant is then subjected to sequential ammonium sulfate precipitation. The fraction containing the polypeptide or protein is subjected to gel filtration in an appropriately sized dextran or polyacrylamide column to separate the proteins. If necessary, the protein fraction may be further purified by HPLC.

This aspect of the present invention also contemplates variants of the protein or polypeptide. Variants may be modified by, for example, the deletion or addition of amino acids that have minimal influence on the properties, secondary structure, and hydropathic nature of the protein or polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

### EXAMPLES

The following examples are intended to illustrate, but by no means are intended to limit, the scope of the present invention as set forth in the appended claims.

### Example 1

### Plant Material

Apple (Malus×domestica) cultivar Gala was used to generate a cDNA prey library for the yeast two-hybrid assay, and the HIPM gene was characterized from it.

Arabidopsis thaliana ecotype Columbia was used as the source of the AtHIPM gene and for transformation of the AtHIPM overexpressing construct. A T3 line, in which T-DNA was inserted in the 5′-UTR of the AtHIPM gene, was obtained from the Arabidopsis Stock Center (Colombus, Ohio, USA), and T4 seeds with homozygosity of the T-DNA-inserted locus, which was determined by PCR with two gene-specific primers, 5′-TTAGATATCCACATAACATGTGC-3′ (SEQ ID NO: 8) and 5′-TTCACAAACATAGCATGACAGG-3′ (SEQ ID NO: 9), and one primer from T-DNA, 5′-TGGTTCACGTAGTGGGCCATCG-3′ (SEQ ID NO: 10), were used for further experiments. Cultivar Galaxy was used for gene silencing. N. benthamiana was used for transient expression experiments.

The rice cultivars Nipponbare and Jefferson were used for cloning OsHIPM, and the cultivar Nipponbare will be used for transformation.

### Example 2

### Plant Assay with E. amylovora

E. amylovora strain Ea273 and its hrpN deletion mutant Ea273ΔhrpN were used to assay virulence of strains in immature pear fruits as described in Oh et al., “The Hrp Pathogenicity Island of Erwinia amylovora and the Identification of Three Novel Genes Required for Systemic Infection,” Mol. Plant Pathol. 6:125-38 (2005), which is hereby incorporated by reference in its entirety. In addition, Ea273 was used to determine whether expression of the HIPM gene is induced by E. amylovora in apple. In this experiment, 5 mM potassium phosphate (pH 6.5) was used as a buffer control.

### Example 3

### Total RNA Isolation, RT-PCR, and Recombinant DNA Techniques

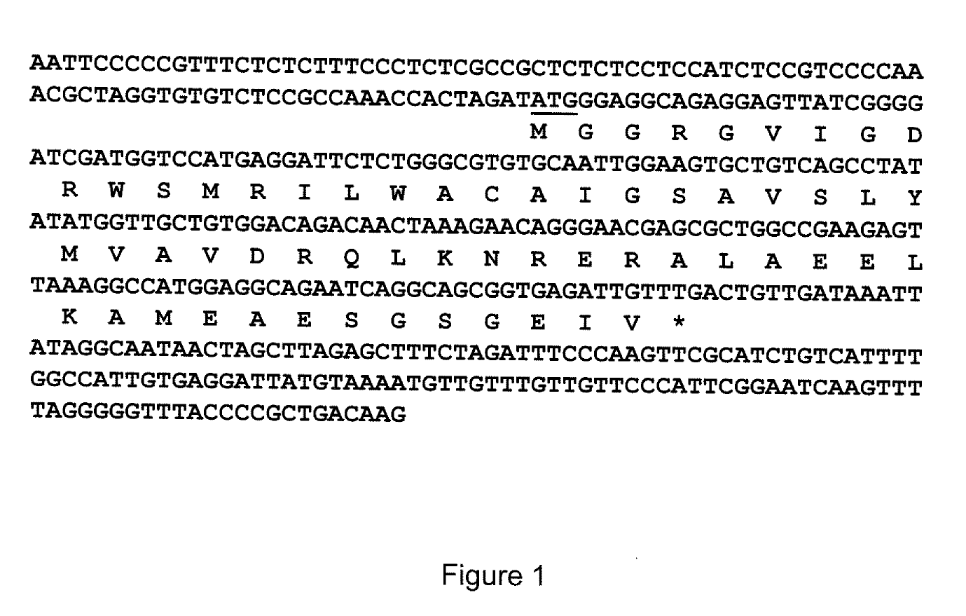

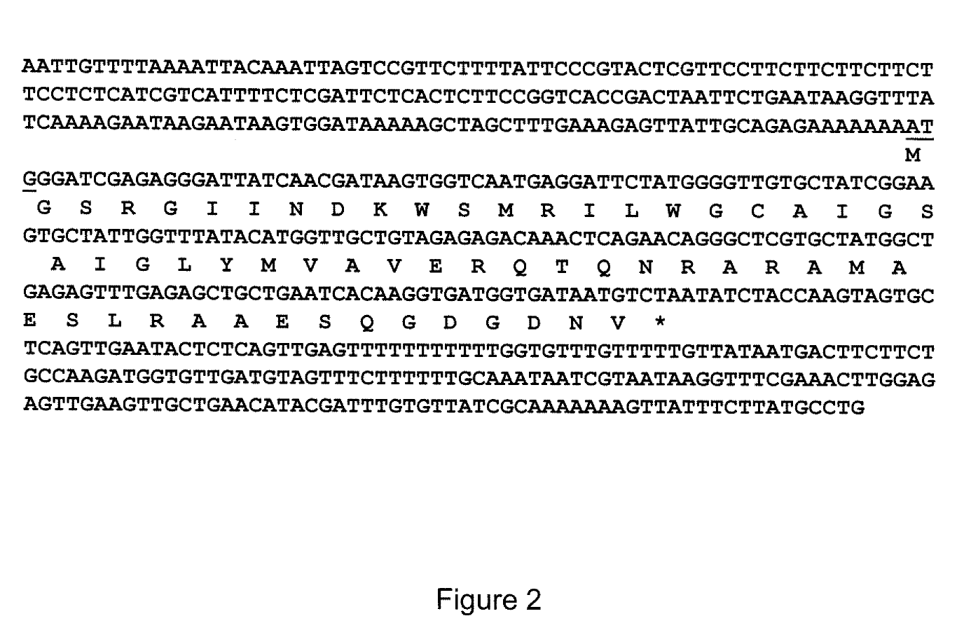

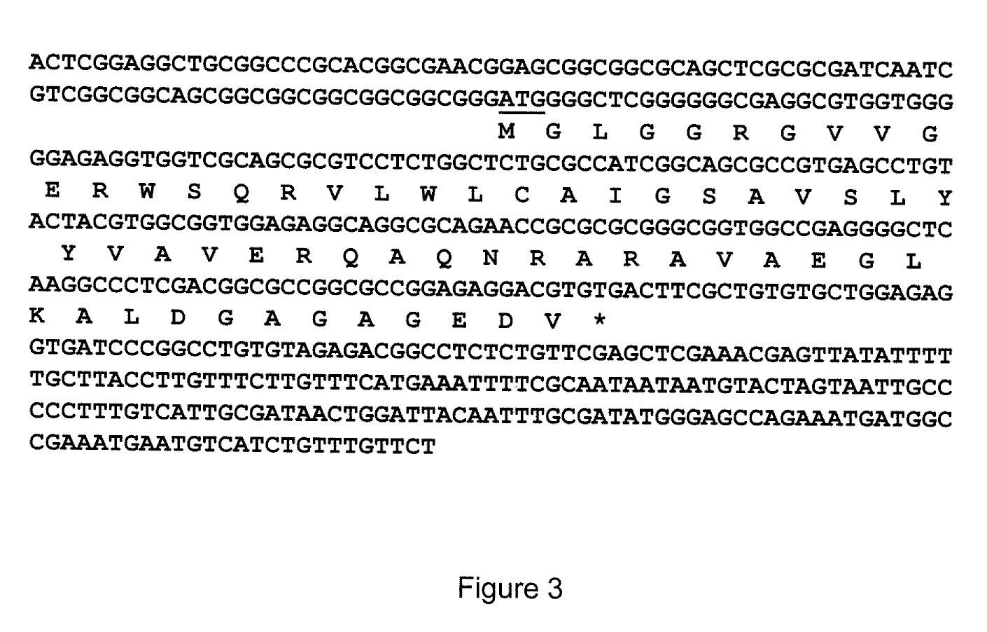

Total RNA was isolated from several parts of apple using the protocol described in Komjanc et al., “A Leucine-rich Repeat Receptor-like Protein Kinase (LRPKm1) Gene Is Induced in Malus×domestica by Venturia inaequalis Infection and Salicylic Acid Treatment,” Plant Mol. Biol. 40:945-57 (1999), which is hereby incorporated by reference in its entirety, and from several parts of Arabidopsis and leaves of rice using RNeasy™ kit (Qiagen, Hilden, Germany). Total RNA concentration was measured using the RiboGreen™ RNA quantitation reagent and kit (Molecular Probes, Eugene, Oreg., USA). RT-PCR was carried out as described in Wilson et al., “Concentration-dependent Patterning of the Xenopus Ectoderm by BMP4 and Its Signal Transducer Smad1,” Development 124:3177-84 (1997), which is hereby incorporated by reference in its entirety, with 0.5-2 μg (HIPM and AtHIPM) or 0.7 μg (OsHIPM) of total RNA. The HIPM nucleotide (SEQ ID NO: 1) and amino acid (SEQ ID NO: 2) sequences are shown in FIG. 1. The AtHIPM nucleotide (SEQ ID NO: 3) and amino acid (SEQ ID NO: 4) sequences are shown in FIG. 2. The Nipponbare OsHIPM nucleotide (SEQ ID NO: 5) and amino acid (SEQ ID NO: 6) sequences are shown in FIG. 3.

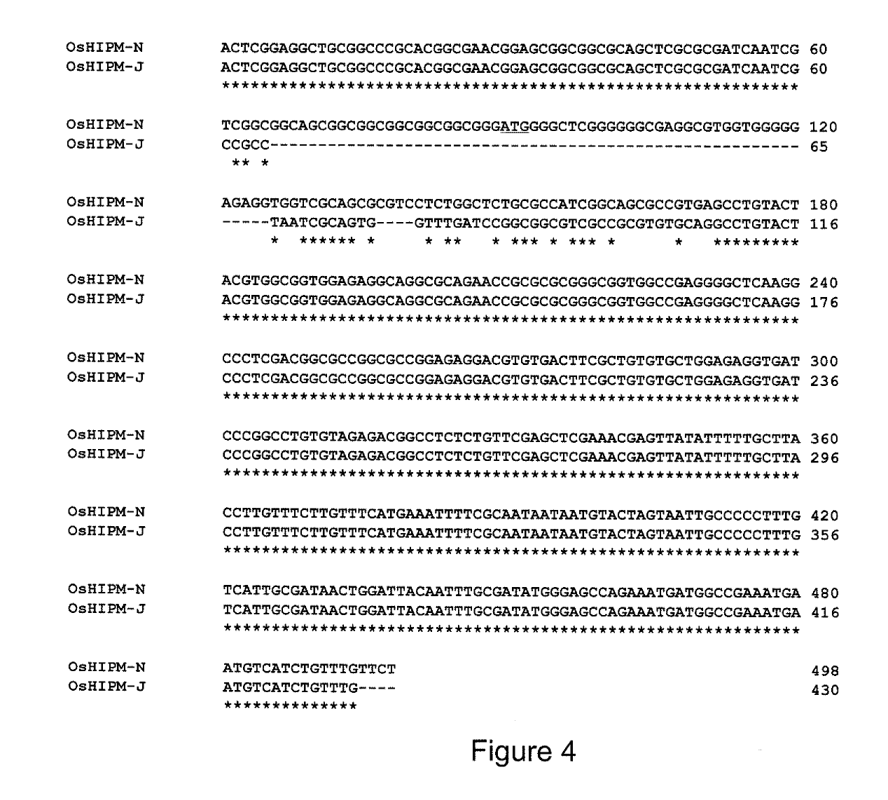

Based on the OsHIPM sequence from the Nipponbare cultivar, the OsHIPM gene locus was cloned by RT-PCR from the Jefferson cultivar of Japonica rice. The gene fragment was smaller than expected: a 430-bp fragment, named OsHIPM-J, was amplified from cDNA generated with total RNA from the Jefferson cultivar. As shown in FIG. 4, when the sequence of the 430-bp fragment (SEQ ID NO: 7) was compared to that of the Nipponbare OsHIPM gene (OsHIPM-N) (SEQ ID NO: 5), three regions, a 60-bp and two 4-bp regions, were absent. Importantly, the 60-bp deletion region contains the start codon of OsHIPM-N, suggesting that OsHIPM-J may be non-functional. In addition, there were several mismatched bases around the first 4-bp deletion region. Except for these regions, the sequence of the cloned fragment from Jefferson was identical to that of the OsHIPM-N gene.

General DNA manipulations, including cloning and plasmid construction, were performed as described in SAMBROOK & RUSSELL, MOLECULAR CLONING (1989), which is hereby incorporated by reference in its entirety. Plasmids were transformed into bacterial strains by electroporation using the Gene Pulser II (Bio-Rad, Richmond, Calif., USA). DNA sequencing was performed at the Cornell University Biotechnology Program DNA Sequencing Facility.

### Example 4

### 5′-RACE

A 5′ RLM-RACE kit (Ambion, Austin, Tex., USA) was used to clone and sequence the 5′ end of the HIPMtranscript. 10 μg of total RNA from the apple cultivar Gala was used to make cDNA. As gene-specific primers, 5′-ACAGCACTTCCAATTGCACACG-3′ (SEQ ID NO: 11) and 5′-CTTTAGTTGTCTGTCCACAGCA-3′ (SEQ ID NO: 12) were used for amplifying the 5′ end of the HIPM transcript.

### Example 5

### Yeast Two-hybrid Assay

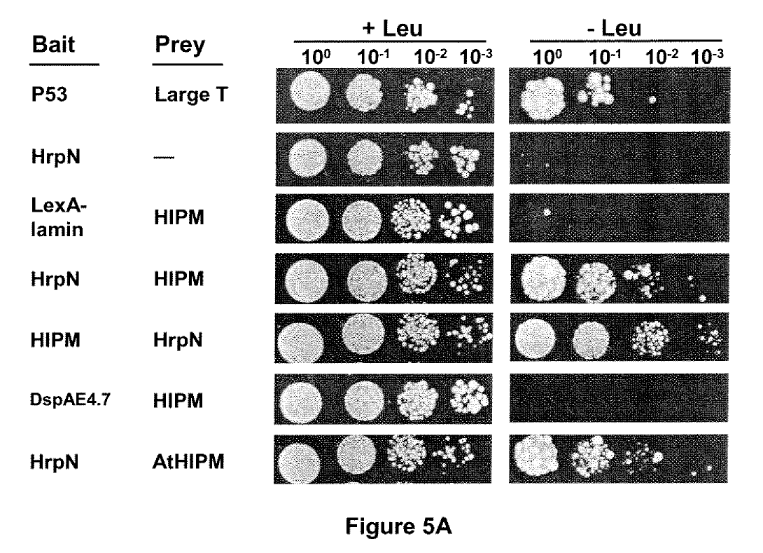

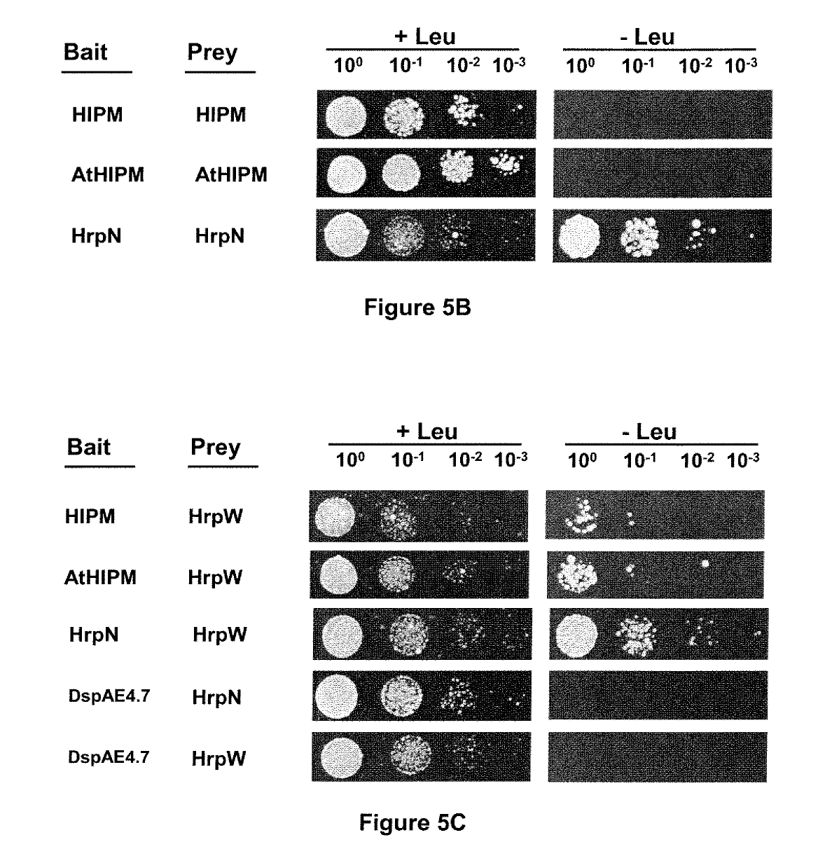

The Matchmaker LexA Two-Hybrid System (Clontech, Palo Alto, Calif., USA) was used for screening HrpN-interacting protein(s) in apple. Briefly, bait in a pGilda vector and prey in a pB42AD vector were co-transformed into Saccharomyces cerevisiae EGY48 [ura3, his3, trp1, LexAop-LEU2, p8op-lacZ] by the LiAc/PEG transformation method (CLONTECH, YEAST PROTOCOLS HANDBOOK (2001), which is hereby incorporated by reference in its entirety). Transformants were selected on minimal synthetic dropout (“SD”) medium containing glucose but lacking histidine and tryptophan (“SD/glu-HT”). To identify yeast transformants with positive interaction, the transformants were screened on SD medium with galactose but without histidine, tryptophan, and leucine (“SD/gal-HTL”). Yeast colonies with positive interaction grow in this medium. A yeast strain transformed with p53 as bait and large antigen T as prey was used as a positive control. LexA-lamin or DspA/E4.7 from the 4.67-kb 5′ end portion of dspA/E (Meng et al., “Apple Proteins That Interact with DspA/E, a Pathogenicity Effector of Erwinia amylovora, the Fire Blight Pathogen,” Mol. Plant-Microbe Interact. 19:53-61 (2006), which is hereby incorporated by reference in its entirety) were used with the proteins indicated in FIGS. 5A-C as negative controls. Yeast cell cultures were grown overnight in the SD/glu-HT medium. The cells then were washed once with water and resuspended in water. 10 μl of yeast cell suspension (OD₆₀₀=0.2) was dropped on SD/gal-HTL plates. As a control, the same yeast suspension was dropped on SD/gal-HT plates with leucine.

### Example 6

### Generation of Bait and Prey Constructs

The full-length hrpN gene was cloned in the pGilda vector by PCR with EcoRI added to the forward primer, 5′-AGGAATTCATGAGTCTGAATACAAGTGC-3′ (SEQ ID NO: 13), and with BamHI added to the reverse primer, 5′-GCGGATCCAAGCTTAAGCCGCGCCCAG-3′ (SEQ ID NO: 14). This clone was called pGilda-hrpN, and was used as bait. A cDNA prey library was generated from total RNA isolated from the apple cultivar Gala in the pB42AD vector with added EcoRI and XhoI sites.

For cloning of hrpN and HIPM into pB42AD and pGilda, pGilda-hrpN and pAD-HIPM were digested with EcoRI and XhoI, and ligated to pB42AD and pGilda, respectively. AtHIPM was amplified by PCR with the forward primer, 5′-CGGAATTCAACGATAAGTGGTCAATGAG (SEQ ID NO: 15), and two reverse primers, 5′-CGGGATCCTTAGACATTATCACCATCACCTTG (SEQ ID NO: 16) and 5′-GCCGCTCGAGGTATTCAACTGAGCACTACTTG (SEQ ID NO: 17), for cloning into pGilda and pB42AD, respectively. The full-length hrp W gene was amplified by PCR and cloned into pGilda and pB42AD vectors.

To remove portions of the HrpN protein from both the N-terminus and C-terminus, four more forward primers and five more reverse primers were used. For deleting 50, 100, 150, 200, 250, or 300 amino acids from the C-terminus, the forward primer with added EcoRI, 5′-AGGAATTCATGAGTCTGAATACAAGTGC-3′ (SEQ ID NO: 13), and the reverse primers with added BamHI, 5′-CGGGATCCTTACTTGGCTTTGTTGAACTGCTC-3′ (SEQ ID NO: 18), 5-CGGGATCCTTAGAACTGACCGATTTCCTTCGC-3′ (SEQ ID NO: 19), 5′-CGGGATCCTTACAGGTTTTGCAGCCCTTTGC-3′ (SEQ ID NO: 20), 5′-CGGGATCCTTAATAGGCGTTCTGCTCGCCTTCG-3′ (SEQ ID NO: 21), and 5′-CGGGATCCTTAGGACGTTGAGTTAATACCCAGC-3′ (SEQ ID NO: 22) were used for PCR. For deleting 50, 100, 150, or 200 amino acids from the N-terminus, the reverse primer with added BamHI, 5′-GCGGATCCAAGCTTAAGCCGCGCCCAG-3′ (SEQ ID NO: 14), and the forward primers with added EcoRI, 5′-CGGAATTCGATACCGTCAATCAGCTG-3′ (SEQ ID NO: 23), 5-CGGAATTCCTGAACGATATGTTAGGC-3′ (SEQ ID NO: 24), 5′-CGGAATTCCAGCTGCTGAAGATGTTCAGC-3′ (SEQ ID NO: 25), and 5′-CGGAATTCAATGCTGGCACGGGTCTTGACG-3′ (SEQ ID NO: 26), were used for PCR. These PCR products were cloned into the pGilda vector.

### Example 7

### In Vitro Pull-down Assay

To determine the interaction between HrpN and HIPM or AtHIPM in vitro, HrpN was tagged with T7 tag in the pET-24a vector, overexpressed in E. coli BL21 (DE3), and purified with T7 Tag Affinity Purification Kit (Novagen, Darmstadt, Germany). Both HIPM and AtHIPM were tagged with FLAG in the pFLAG-CTC vector (Scientific Imaging Systems, Rochester, N.Y., USA) and overexpressed in E. coli BL21 (DE3). Cell lysate with 5 μg of T7-HrpN was incubated with 50 μl of T7 tag antibody agarose beads at room temperature for 30 minutes with shaking. Beads were washed thrice more with T7 Tag bind/wash buffer, then incubated with cell lysate with 5 μg of HIPM-FLAG or AtHIPM-FLAG at room temperature for one hour, and washed four times more with T7 Tag bind/wash buffer. 150 μl of elution buffer were added in the pellet and proteins were eluted from the T7 tag antibody agarose beads. This step was repeated once more, and 45 μl of neutralization buffer were added in the protein solution. 18 μl of the protein solution were resuspended in 6 μl of 4×SDS-PAGE loading buffer.

### Example 8

### Immunoblotting

Protein samples were denatured by holding them in boiling water for five minutes, electrophoresed on a 4-20% gradient of SDS-PAGE gel (Gradipore, Frenchs Forest, Australia), and transferred to PVDF (Immobilon™-P Millipore Corp., Bedford, Mass., USA) by a semi-dry electroblotting method (Gravel & Golaz, “Protein Blotting by the Semidry Method,” in THE PROTEIN PROTOCOLS HANDBOOK 249-60 (John M. Walker ed., 2d ed. 1996), which is hereby incorporated by reference in its entirety). Western blotting was performed with the Western-Star™ protein detection kit (TROPIX, Bedford, Mass., USA). The HrpN antibody described previously (Wei et al., “Harpin, Elicitor of the Hypersensitive Response Produced by the Plant Pathogen Erwinia amylovora,” Science 257:85-8 (1992), which is hereby incorporated by reference in its entirety) and the FLAG M2 antibody (Novagen, Darmstadt, Germany) were used to detect T7-HrpN and HIPM-FLAG or AtHIPM-FLAG, respectively. To detect bait and prey proteins, LexA monoclonal antibody (Clontech, Palo Alto, Calif., USA) and hemagglutinin (HA) polyclonal antibody (Rockland, Gilbertsville, Pa., USA) were used, respectively.

### Example 9

### Yeast-Based Signal Peptide Trap System

The yeast-based signal peptide trap system was used to determine whether putative signal peptides are functional as first described in Yamane et al., “A Coupled Yeast Signal Sequence Trap and Transient Plant Expression Strategy to Identify Genes Encoding Secreted Proteins from Peach Pistils,” J. Exp. Bot. 56:2229-38 (2005), which is hereby incorporated by reference in its entirety. Briefly, the full-length cDNAs of the HIPM and AtHIPM genes were fused in frame with the suc2 gene lacking its signal peptide in the pYSST0 vector. In addition, truncated forms of both genes, in which a portion encoding the first 20 amino acids had been deleted, were cloned in the same vector. These constructs were transformed into yeast strain DBYα2445 (Matα, suc2Δ-9, lys2-801, ura3-52, ade2-101) by the Li/PEG transformation method. The growth of the yeast transformants was determined in a sucrose selection medium (1% yeast extract, 2% peptone, 2% sucrose, 2% agar).

### Example 10

### Transient Expression in Nicotiana benthamiana

The full-length HIPM and AtHIPM genes were fused in frame with soluble-modified green fluorescent protein (“smGFP”) in pCAMBIA2300-smGFP (Davis & Vierstra, “Soluble, Highly Fluorescent Variants of Green Fluorescent Protein (GFP) for Use in Higher Plants,” Plant Mol. Biol. 36:521-8 (1998), which is hereby incorporated by reference in its entirety) for confocal microscopy. These constructs, as well as pCAMBIA2300-smGFP as a control, were separately transformed into Agrobacterium tumefaciens strain GV2260. Transient expression was carried out as described in Abramovitch et al., “Pseudomonas Type III Effector AvrPtoB Induces Plant Disease Susceptibility by Inhibition of Host Programmed Cell Death,” EMBO J. 22:60-9 (2003), which is hereby incorporated by reference in its entirety.

### Example 11

### Confocal Microscopy

Leaf discs were harvested 24 hours after agroinfiltration, and GFP signals were observed with a BioRad Leica MRC-600 confocal microscope (BioRad Biosciences, Hercules, Calif., USA) using an HC PL APO 20× oil immersion objective and the 488 nm and 543 nm lines generated by argon lasers at Cornell Biotechnology Resource Center. Emission window ranges for GFP and chlorophyll were 500-580 nm and 634-718 nm, respectively. After GFP signals were captured, three sections were overlaid in a single image, and images were saved with a resolution of 512×512 pixels.

### Example 12

### Gene Silencing Constructs

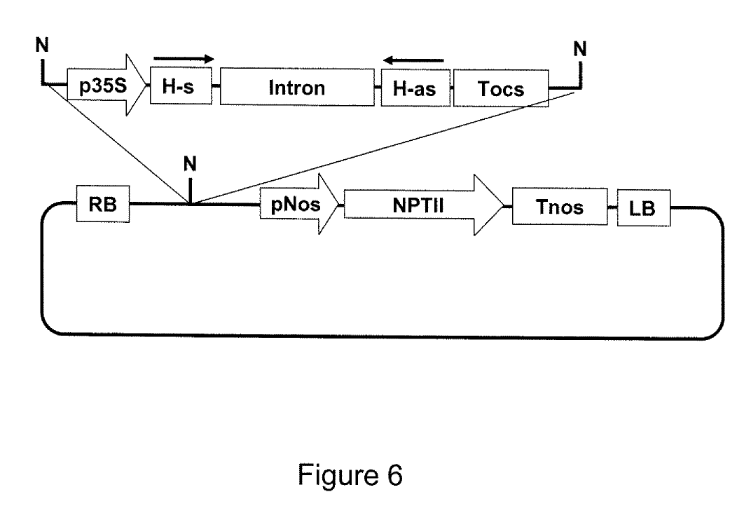

To make a hairpin loop structure of the HIPM gene for gene silencing, the pHANNIBAL cloning system was used. The full length sense (see Table 1) and anti-sense of the HIPM gene were cloned into a pHANNIBAL vector under control of the 35S promoter and the octopine synthetase terminator. This whole fragment (cut with NotI restriction enzyme) was transferred into vector pART27, named pART27-Hs-Has, illustrated in FIG. 6. The plasmid was transformed into A. tumefaciens strain EHA105 for apple transformation.

TABLE 1

Gene Silencing Constructs

| | Gene | Ab- | |
|---|---|---|---|
| Con- | Si- | brevi- | Sense Sequence Cloned into |
| struct | lenced | ation | Construct |
| pART27- | HIPM | hpH | ATGGGAGGCAGAGGAGTTATCGGGGAT |
| Hs-Has | | (hair- | CGATGGTCCATGAGGATTCTCTGGGCGT |
| | | pin | GTGCAATTGGAAGTGCTGTCAGCCTATA |
| | | con- | TATGGTTGCTGTGGACAGACAACTAAAG |
| | | struct | AACAGGGAACGAGCGCTGGCCGAAGAG |
| | | of | TTAAAGGCCATGGAGGCAGAATCAGGC |
| | | HIPM) | AGCGGTGAGATTGTTTG |
| | | | (SEQ ID NO: 27) |
| pART27- | AtHIPM | hpAtH | ATGGGATCGAGAGGGATTATCAACGAT |
| AtHs- | | | AAGTGGTCAATGAGGATTCTATGGGGTT |
| AtHas | | | GTGCTATCGGAAGTGCTATTGGTTTATA |
| | | | CATGGTTGCTGTAGAGAGACAAACTCAG |
| | | | AACAGGGCTCGTGCTATGGCTGAGAGTT |
| | | | TGAGAGCTGCTGAATCACAAGGTGATGG |
| | | | TGATAATGTCT |
| | | | (SEQ ID NO: 28) |
| pANDA- | OsHIPM | hpOsH1 | ACTCGGAGGCTGCGGCCCGCACGGCGA |
| OsH1 | | | ACGGAGCGGCGGCGCAGCTCGCGCGAT |
| | | | CAATCGCCGCCTAATCGCAGTGGTTTGA |
| | | | TCCGGCGGCGTCGCCGCGTGTGCAGGCC |
| | | | TGTACTACGTGGCGGTGGAGAGGCAGG |
| | | | CGCAGAACCGCGCGCGGGCGGTGGCCG |
| | | | AGGGGCTCAAGGCCCTCGACGGCGCCG |
| | | | GCGCCGGAGAGGACGTGTGACTTCGCTG |
| | | | TGTGCTGGAGAGGTGATCCCGGCCTGTG |
| | | | TAGAGACGGCCTCTCTGTTCGAGCTCGA |
| | | | AACGAGTTATATTTTTGCTTACCTTGTTT |
| | | | CTTGTTTCATGAAATTTTCGCAATAATA |
| | | | ATGTACTAGTAATTGCCCCCTTTGTCATT |
| | | | GCGATAACTGGATTACAATTTGCGATAT |
| | | | GGGAGCCAGAAATGATGGCCGAAATGA |
| | | | ATGTCATCTGTTTG |
| | | | (SEQ ID NO: 29) |
| pANDA- | OsHIPM | hpOsH2 | GTGGCGGTGGAGAGGCAGGCGCAGAAC |
| OsH2 | | | CGCGCGCGGGCGGTGGCCGAGGGGCTC |
| | | | AAGGCCCTCGACGGCGCCGGCGCCGGA |
| | | | GAGGACGTGTGACTTCGCTGTGTGCTGG |
| | | | AGAGGTGATCCCGGCCTGTGTAGAGACG |
| | | | GCCTCTCTGTTCGAGCTCGAAACGAGTT |
| | | | ATATTTTTGCTTACCTTGTTTCTTGTTTC |
| | | | ATGAAATTTTCGCAATAATAATGTACTA |
| | | | GTAATTGCCCCCTTTGTCATTGCGATAA |
| | | | CTGGATTACAATTTGCGATATGGGAGCC |
| | | | AGAAATGATGGCCGAAATGAATGTCATC |
| | | | TGTTTG |
| | | | (SEQ ID NO: 30) |

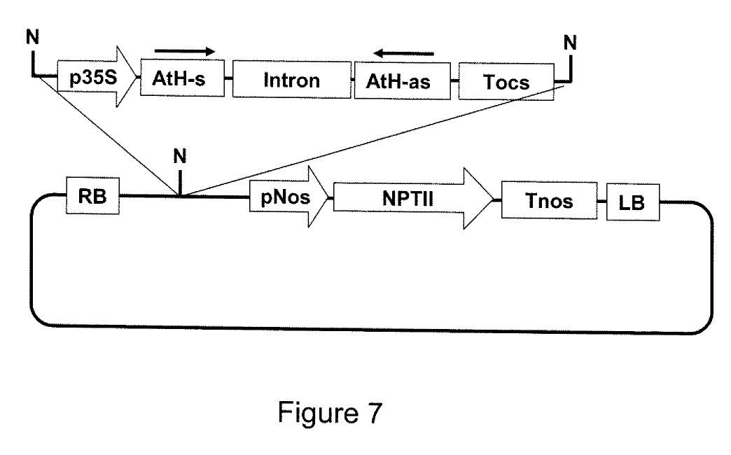

To make a construct for AtHIPM gene silencing, the full-length sense (see Table 1) and anti-sense of the AtHIPM gene were cloned into a pHANNIBAL vector under control of the 35S promoter and the octopine synthetase terminator. This fragment was transferred into vector pART27, and named pART27-AtHs-AtHas, illustrated in FIG. 7.

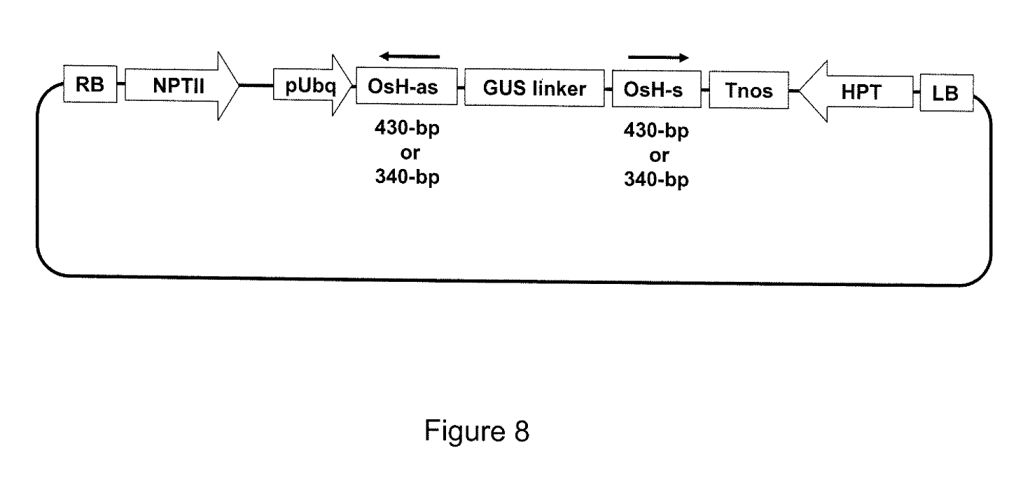

To determine whether OsHIPM silencing increases plant growth and grain yield in rice, two OsHIPM silencing constructs were generated. Because the 430-bp fragment of the OsHIPM gene from the Jefferson cultivar does not contain the start codon and its nucleotide sequence is highly conserved relative to OsHIPM-N, this fragment was used to develop silencing constructs. Using the Gateway cloning system (Invitrogen), the whole 430-bp fragment and a 312-bp fragment from the 3′-end were cloned into a pENTR vector (Invitrogen) under control of the maize ubiquitin promoter and the nopoline synthetase terminator. These fragments were then transferred separately by the LR clonase reaction into pANDA, a vector developed for and shown to work well in rice (Miki & Shimamoto, “Simple RNAi Vectors for Stable and Transient Suppression of Gene Function in Rice,” Plant Cell Physiol. 45(4):490-5 (2004), which is hereby incorporated by reference in its entirety), to develop two RNAi constructs (named pANDA-OsH1 and pANDA-OsH2) configured as shown in FIG. 8. The nucleotide sequences of the “OsH-s” portion of these constructs are set forth in Table 1. “OsH-as” is the anti-sense sequence of the respective OsH-s sequence. These silencing constructs will be transformed into the Nipponbare cultivar to generate transgenic rice lines.

### Example 13

### Overexpression Constructs

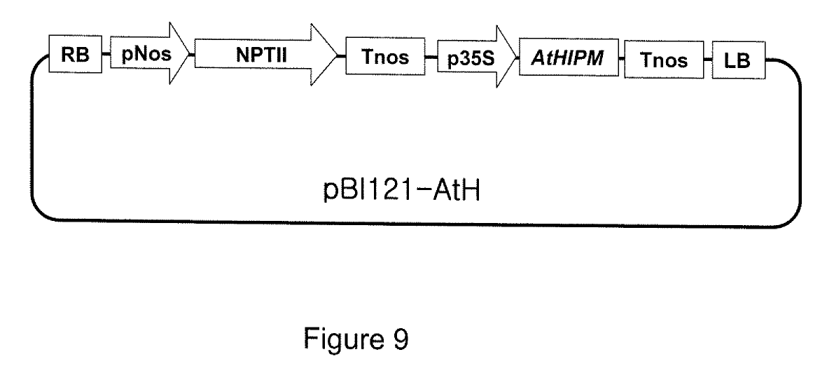

To make a construct for overexpressing AtHIPM, the full length AtHIPM gene (see Table 2) was cloned into a pBI121 vector under control of the 35S promoter and the Tnos terminator, and named pBI121-AtH, illustrated in FIG. 9.

TABLE 2

Overexpression Constructs

| | Gene | | |
|---|---|---|---|
| | Over- | | Sequence Cloned into |
| Construct | expressed | Abbreviation | Construct |
| pBI121-H | HIPM | OxH | ATGGGAGGCAGAGGAGTTATCGGGGAT |
| | | | CGATGGTCCATGAGGATTCTCTGGGCGT |
| | | | GTGCAATTGGAAGTGCTGTCAGCCTATA |
| | | | TATGGTTGCTGTGGACAGACAACTAAAG |
| | | | AACAGGGAACGAGCGCTGGCCGAAGAG |
| | | | TTAAAGGCCATGGAGGCAGAATCAGGC |
| | | | AGCGGTGAGATTGTTTGA |
| | | | (SEQ ID NO: 31) |
| pBI121- | AtHIPM | OxAtH | ATGGGATCGAGAGGGATTATCAACGAT |
| AtH | | | AAGTGGTCAATGAGGATTCTATGGGGTT |
| | | | GTGCTATCGGAAGTGCTATTGGTTTATA |
| | | | CATGGTTGCTGTAGAGAGACAAACTCAG |
| | | | AACAGGGCTCGTGCTATGGCTGAGAGTT |
| | | | TGAGAGCTGCTGAATCACAAGGTGATGG |
| | | | TGATAATGTCTAA |
| | | | (SEQ ID NO: 32) |
| pCAMBIA | OsHIPM | OxOsH | ATGGGGCTCGGGGGGCGAGGCGTGGTG |
| 1300-OsH | | | GGGGAGAGGTGGTCGCAGCGCGTCCTCT |
| | | | GGCTCTGCGCCATCGGCAGCGCCGTGAG |
| | | | CCTGTACTACGTGGCGGTGGAGAGGCAG |
| | | | GCGCAGAACCGCGCGCGGGCGGTGGCC |
| | | | GAGGGGCTCAAGGCCCTCGACGGCGCC |
| | | | GGCGCCGGAGAGGACGTGTGACT |
| | | | (SEQ ID NO: 33) |

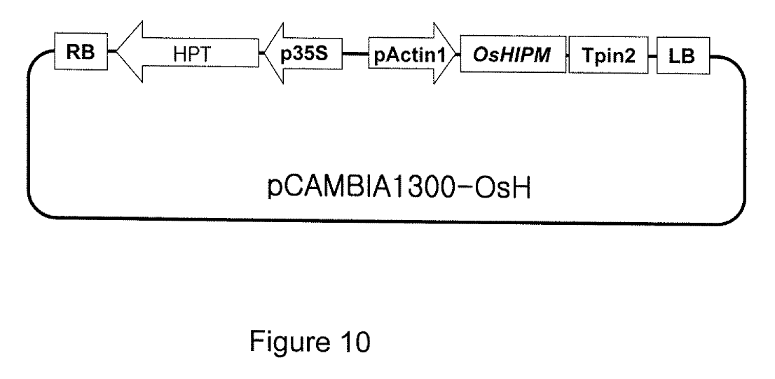

The full-length OsHIPM gene (see Table 2) will be cloned by RT-PCR, using total RNA isolated from panicles of the Nipponbare cultivar. The gene, driven by the rice Actin1 promoter, will be cloned into a pCAMBIA1300 vector to overexpress the OsHIPM gene in rice, as illustrated in FIG. 10.

Constructs for overexpressing HIPM may be produced, for example, as described above for the AtHIPM overexpression construct using the full-length HIPM gene (see Table 2).

### Example 14

### Arabidopsis Transformation for AtHIPM Overexpression

To make AtHIPM overexpressing Arabidopsis plants, the full-length AtHIPM gene was cloned in the pBI121 vector, named BI121-AtHIPM. The floral dipping method was used for Arabidopsis transformation (Clough & Bent, “Floral Dip: A Simplified Method for Agrobacterium-mediated Transformation of Arabidopsis thaliana,” Plant J. 16:735-43 (1998), which is hereby incorporated by reference in its entirety). Briefly, Arabidopsis plants with closed flowers were dipped into a bacterial suspension (OD₆₀₀=0.2) of A. tumefaciens strain GV3101 with pBIl21-AtHIPM or pBI121 in a 5% glucose solution with 0.04% silwet. Those plants were placed in a growth room at 22° C. and illuminated for 16 hours per day. After harvesting T1 seeds, T1 transgenic seedlings were selected in 1×MS medium with 50 μg/ml of kanamycin. Kanamycin-resistant T1 seedlings were sown in pots for harvest of T2 seeds. About 10 different kanamycin-resistant T2 seedlings were planted to produce T3 seeds from which homozygous seeds were selected. For further experiments, homozygous T3 plants were used.

### Example 15

### Root and Top Growth Measurements in Arabidopsis

For measurement of root growth, around 30 seeds of Arabidopsis, previously held at 4° C. for 5 days, were lined up on two plates of 0.5×MS medium containing 3% sucrose. For determining the effect of HrpN, seeds were soaked in a 0.075% agarose solution with 0.5 mg/ml of Messenger® (15 μg/ml of HrpN) (Eden Bioscience, Bothell, Wash., USA). The seeded plates were placed vertically in a growth room at 22° C. and illuminated for 14 hours per day. After 10 days, root length was measured. For top growth, cold-treated seeds were planted in 2.5-inch (6.3 cm) rectangular pots and grown for 3 weeks. Messenger® (0.5 mg/ml) or water (as the control treatment) was sprayed once on the plants to runoff. The lengths of the three longest leaves per plant were measured one week after spraying.

### Example 16

### Determination of Responses to Plant Hormones in Arabidopsis

For methyl jasmonate (“MeJA”) and auxin, around 30 cold-treated seeds of Arabidopsis were lined up on two plates of 0.5×MS medium containing 1% sucrose and MeJA or 2,4-D at 0, 1, or 5 μM. The plates were placed vertically in a growth room at 22° C. with 14 hours of light per day. Root length was measured after 10 days. 10 μM of ACC was added in 0.5×MS medium to simulate the presence of ethylene. Plates were kept vertically in the dark for 3 days, and the triple response was determined.

### Example 17

### HIPM and its Ortholog AtHIPM Interact with HrpN, a Harpin of E. amylovora

Apple proteins that interact with HrpN, the archetype harpin of E. amylovora, were screened with a yeast two-hybrid system. A cDNA prey library from the apple cultivar Gala was screened with the full-length HrpN protein as bait. Of more than 106 primary yeast transformants screened, 24 positive clones were selected initially. Those 24 prey clones were isolated from yeast, sequenced, and re-transformed into yeast harboring the hrpN gene cloned in a bait vector. On retesting, 23 of the clones exhibited negative phenotypes for interaction, while one positive clone, designated “HIPM (HrpN-interacting protein from Malus),” was found to interact with HrpN, as shown in FIG. 5A.

Two proteins, LexA-lamin and DspA/E4.7, encoded by the 4.67-kb 5′ end portion of dspA/E gene of E. amylovora, were chosen to determine whether HrpN-HIPM interaction is specific. As shown in FIG. 5A, HIPM did not interact with either protein, indicating that HrpN-HIPM interaction is specific.

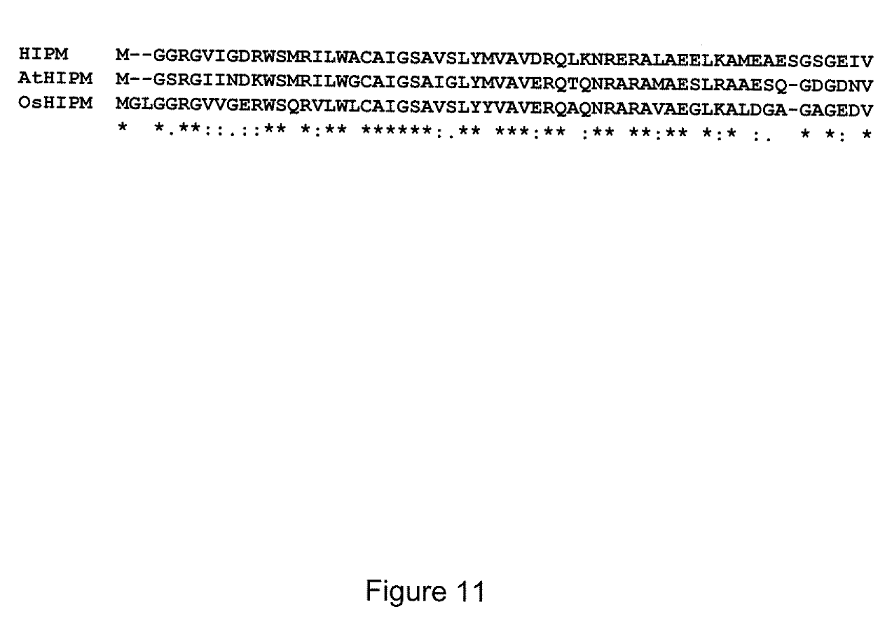

The positive clone contained 314-bp of cDNA of the HIPM transcript, which may encode only the 53 C-terminal amino acids of HIPM. Because the positive clone did not contain the full length HIPM gene, the 5′ end of the gene was amplified and cloned using the 5′ RACE kit. The HIPM gene from apple (SEQ ID NO: 1) encodes a 60 amino acid protein of around 6.5 kDa (SEQ ID NO: 2), as shown in FIG. 1. Two orthologs, At3g15395-1 and XP_{—}464477, designated AtHIPM and OsHIPM, respectively, were found in the genome databases of Arabidopsis and rice. AtHIPM (SEQ ID NO: 3) and OsHIPM-N (SEQ ID NO: 5) encode 6.4 kDa proteins, which consist of 59 and 61 amino acids, respectively, as shown in FIG. 2 (AtHIPM (SEQ ID NO: 4)) and FIG. 3 (OSHIPM-N (SEQ ID NO: 6)). As shown in FIG. 11, at the amino acid level HIPM (SEQ ID NO: 2) is 66% identical to AtHIPM (SEQ ID NO: 4) and 65% identical to OsHIPM-N (SEQ ID NO: 6), while AtHIPM (SEQ ID NO: 4) is 58% identical to OsHIPM-N (SEQ ID NO: 6). At the nucleic acid level, HIPM is 67% identical to AtHIPM and 62% identical to OsHIPM-N, while AtHIPM is 54% identical to OsHIPM-N.

To determine whether AtHIPM also interacts with HrpN in yeast, a fragment of the AtHIPM gene encoding 52 amino acids, which is the region orthologous to the original HIPM prey clone, was cloned into the prey vector and co-transformed with the hrpN gene as bait. AtHIPM also interacted with HrpN in yeast, as shown in FIG. 5A. In addition, whether HrpN, HIPM, or AtHIPM exhibit self-interaction was determined in yeast. However, as shown in FIG. 5B, no self-interaction of HIPM and AtHIPM was detected, but HrpN showed strong self-interaction.

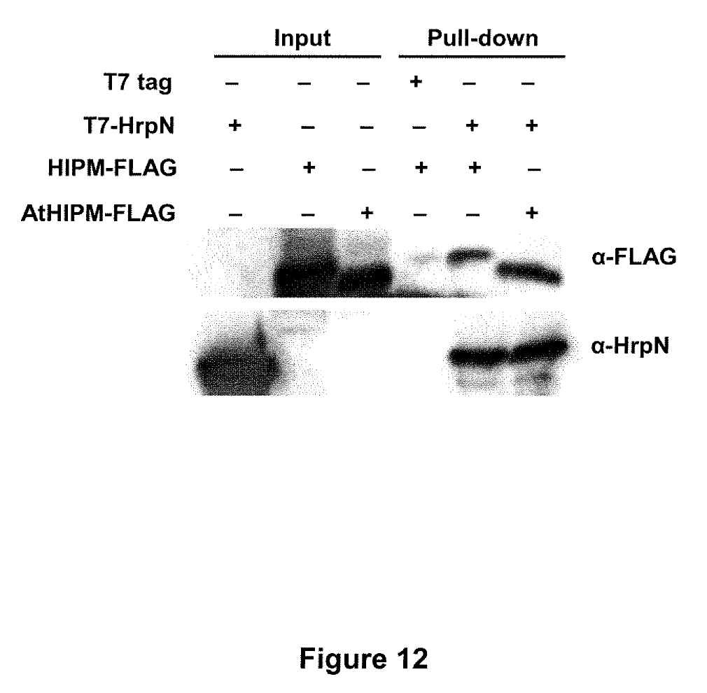

An in vitro pull-down assay was carried out to confirm the interaction of HrpN and HIPM or AtHIPM in vitro. Purified HrpN fused with the T7 tag was pulled-down with T7 tag antibody-linked agarose beads after mixing with HIPM-FLAG or AtHIPM-FLAG protein. As shown in FIG. 12, HIPM-FLAG or AtHIPM-FLAG was detected only when it was pulled down with T7-HrpN, indicating that the proteins interact in vitro.

### Example 18

### HrpW, a Second Harpin of E. amylovora, Also Interacts with HIPM and AtHIPM

E. amylovora produces a second harpin, HrpW, which has a putative pectate lyase domain (Gaudriault et al., “HrpW of Erwinia amylovora, a New Hrp-secreted Protein,” FEBS Lett. 428:224-8 (1998); Kim & Beer, “HrpW of Erwinia amylovora, a New Harpin That Contains a Domain Homologous to Pectate Lyases of a Distinct Class,” J. Bacteriol. 180:5203-10 (1998), which are hereby incorporated by reference in their entirety). HrpW is secreted through the Hrp T3SS, and it may be localized in the intercellular spaces of plant tissues like HrpN. Interestingly, as shown in FIG. 5C, HrpW interacted with both HIPM and AtHIPM in yeast. The fact that both HrpN and HrpW interact with both HIPM and AtHIPM, and HrpN interacts with itself, suggested that HrpW interacts with HrpN. As shown in FIG. 5C, HrpW was found to strongly interact with HrpN in yeast. However, neither HrpN nor HrpW were found to interact with DspA/E4.7.

### Example 19

### The 198 Amino Acid N-terminal Region of HrpN Is Required for Interaction with HIPM

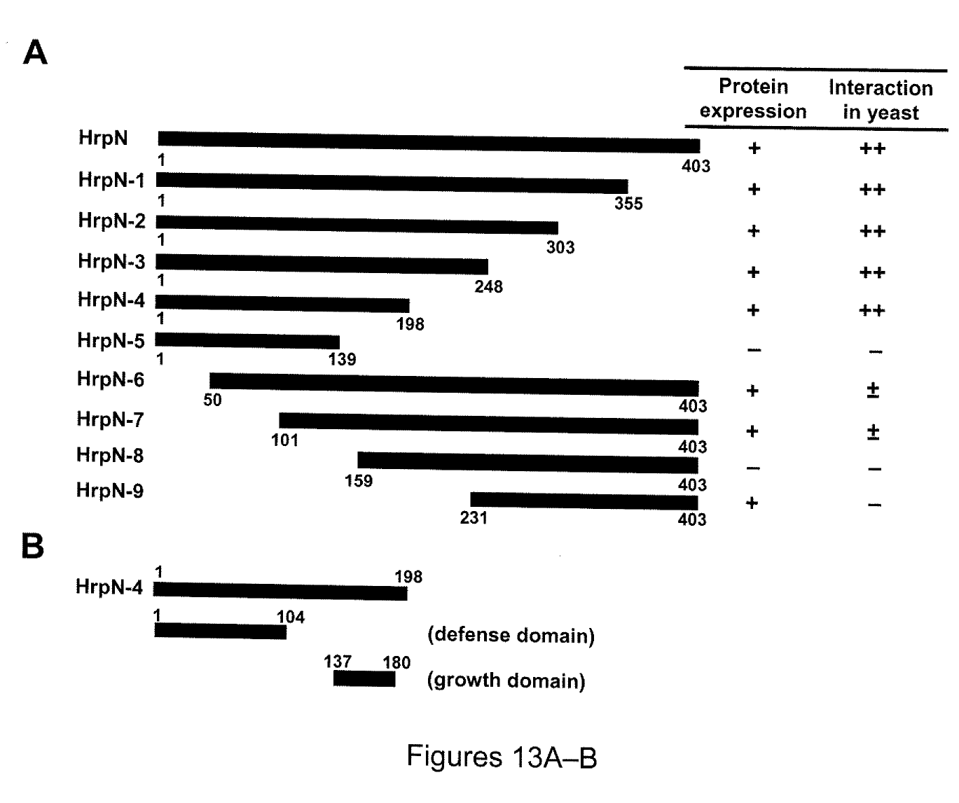

To identify the domain of HrpN that interacts with HIPM, nine truncated derivatives of HrpN were generated as shown in FIG. 13A. First, their expression or stability in yeast was determined using the LexA antibody. Six of the nine derivatives were expressed in yeast (FIG. 13A). Secondly, whether interaction with HIPM occurred was determined in yeast. The 198 aa N-terminal region of HrpN spanning residues 1-198 (“HrpN-4” in FIG. 13A) was needed for full interaction with HIPM, although there was very weak interaction with the the truncated derivative spanning residues 50-403 (“HrpN-6” in FIG. 13A) and that spanning residues 101-403 (“HrpN-7” in FIG. 13A).

Two domains of HrpN, a defense domain (residues 1-104) and a growth domain (residues 137-180), had been identified based on their activities in inducing defense or enhancing growth. Interestingly, as shown in FIG. 13B, these two domains are both located within the 198 aa N-terminal region of the HrpN protein, the same portion that is needed for interaction with HIPM.

To determine whether the 198 aa N-terminal region of HrpN is sufficient for the wild-type level of virulence in host plants, the plasmid carrying DNA encoding the 198 aa N-terminal region of HrpN with its indigenous hrp promoter was transformed into the hrpN deletion mutant of E. amylovora strain Ea273. Virulence of the strain and of appropriate control strains was determined in immature pear fruits. The complemented strain failed to restore virulence in immature pear fruits, indicating that the 198 aa N-terminal region of HrpN is not sufficient for virulence activity of HrpN.

### Example 20

### Both HIPM and AtHIPM Have Functional Signal Peptides

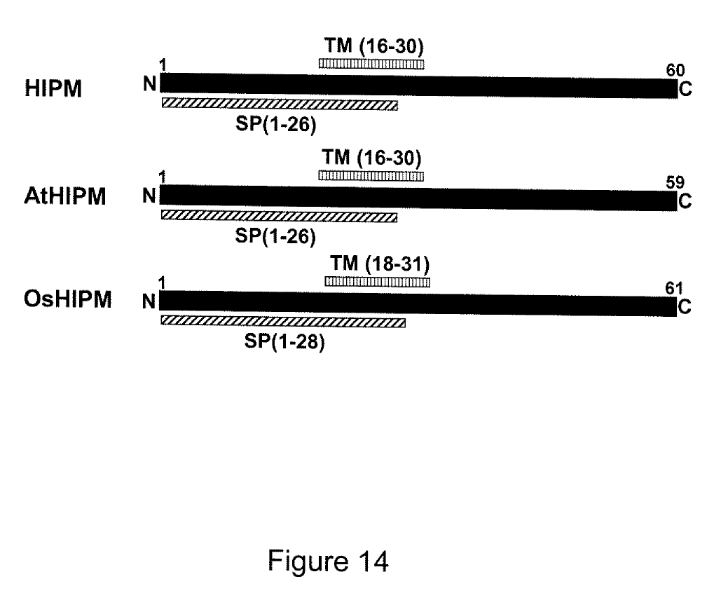

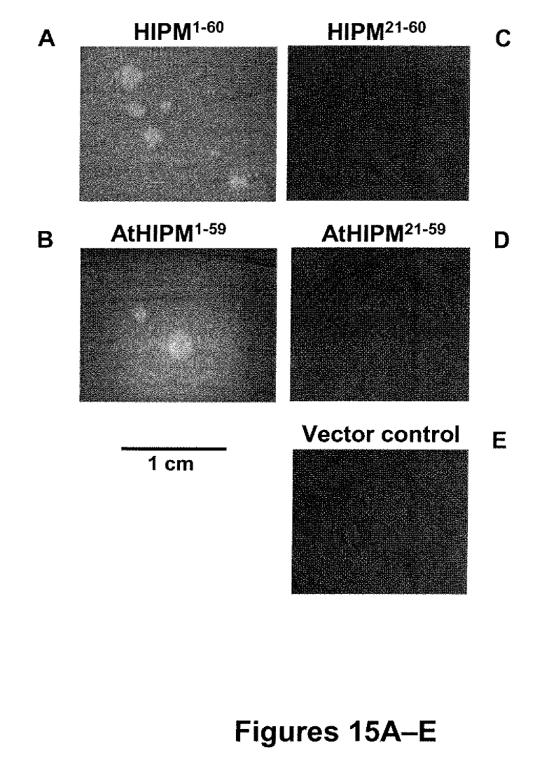

A domain search of two databases (the SignalP 3.0 server for signal peptide (“SP”) prediction and the EXPASy server for transmembrane (“TM”) domain prediction) resulted in the identification of putative SP and TM domains in HIPM, AtHIPM, and OsHIPM-N, as shown in FIG. 14. To determine whether the putative SPs in both HIPM and AtHIPM are functional, the yeast-based SP trap method, which is based on lack of growth of the yeast suc2 mutant in a sucrose medium, was employed. HIPM and AtHIPM were fused with the suc2 genome lacking the DNA region encoding its own SP, and these constructs were put into the yeast suc2 mutant. As shown in FIGS. 15A-E, the suc2 mutant grew in the sucrose medium with the full-length cDNAs of both HIPM (“HIPM¹⁻⁶⁰”) and AtHIPM (“AtHIPM¹⁻⁵⁹”), but not with the truncated forms (i.e., residues 21-60 of HIPM (“HIPM^{21 60}”) and residues 21-59 of AtHIPM (“AtHIPM^{21 59}”)), which lack 20 amino acids at the N-terminal end. These results indicate that HIPM and AtHIPM have functional SPs in their N-termini, and as a consequence, the proteins may be secreted or be embedded in plasma membranes. Because OsHIPM-N has similar domains as HIPM, its SP may also be functional, and it may associate with plasma membranes in rice like HIPM does in apple.

### Example 21

### Both HIPM and AtHIPM Associate, in Clusters, with Plasma Membranes

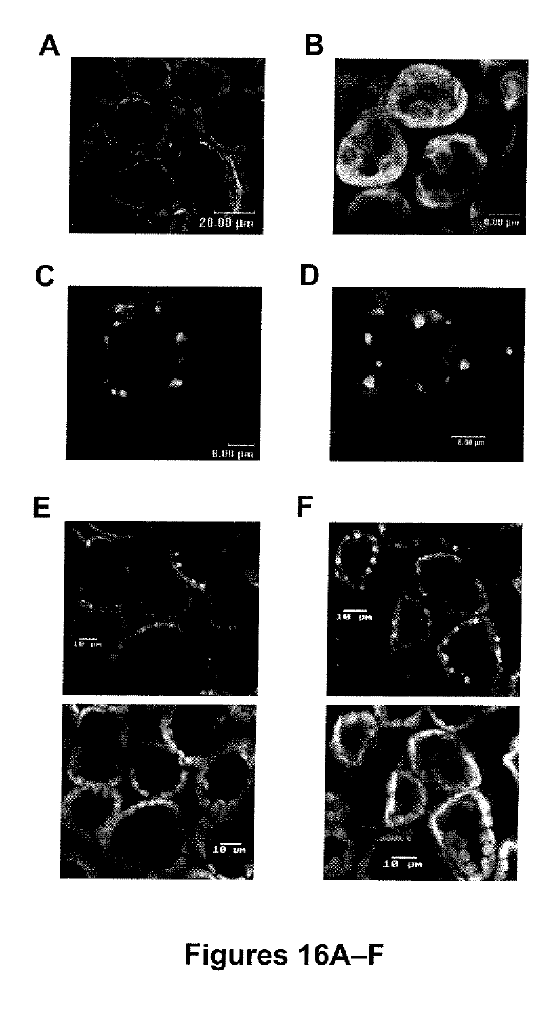

Although both HIPM and AtHIPM were shown to have SPs that are functional in a yeast system, the location of HIPM and AtHIPM in plant cells was not clear. To address the question of location, the GFP gene, whose expression was driven by the 35S promoter, was fused with HIPM and AtHIPM. HIPM-GFP, AtHIPM-GFP, and GFP itself as a control, were transiently expressed in leaves of Nicotiana benthamiana by agroinfiltration. Green fluorescence was observed with confocal microscopy 24 hours after agroinfiltration. Green autofluorescence was observed in the intercellular space of untransformed plants, as shown in FIG. 16A. As shown in FIGS. 16A-E, unlike the GFP control (FIG. 16B), in which green fluorescence was seen throughout the cytoplasm, green fluorescence from both HIPM-GFP (FIGS. 16C and 16E (top)) and AtHIPM-GFP (FIG. 16D) accumulated as large spots coincident with plasma membranes. In addition, in a 0.8 M mannitol solution, cell shapes were irregular due to plasmolysis (FIG. 16F (top)) unlike the round shapes observed in water (FIG. 16E (bottom)), and green fluorescence from HIPM-GFP remained coincident with plasma membranes, as shown in FIG. 16F (top), and did not appear to be cell wall- or plasmodesmata-localized. These results indicate that both HIPM and AtHIPM have functional SPs and both proteins ultimately localize to plasma membranes.

### Example 22

### Both HIPM and AtHIPM Are Expressed More Strongly in Flowers than in Stems and Leaves

HIPM was found in apple, which is a host of E. amylovora. Flowers, vigorously growing young leaves, and shoot tips are the important infection courts for E. amylovora (FIRE BLIGHT (Joel L. Vanneste ed., 2000), which is hereby incorporated by reference in its entirety). To determine the expression pattern of the HIPM gene in apple, total RNA was isolated from leaves, shoots, and flowers at four stages of development: tight cluster (“TC”), pink (“P”), full bloom (“F”), and 6 days after full bloom (“6F”) (Chapman & Catlin, “Growth Stages in Fruit Trees—From Dormant to Fruit Set,” N.Y. Food Life Sci. Bull. 58 (1976), which is hereby incorporated by reference in its entirety).

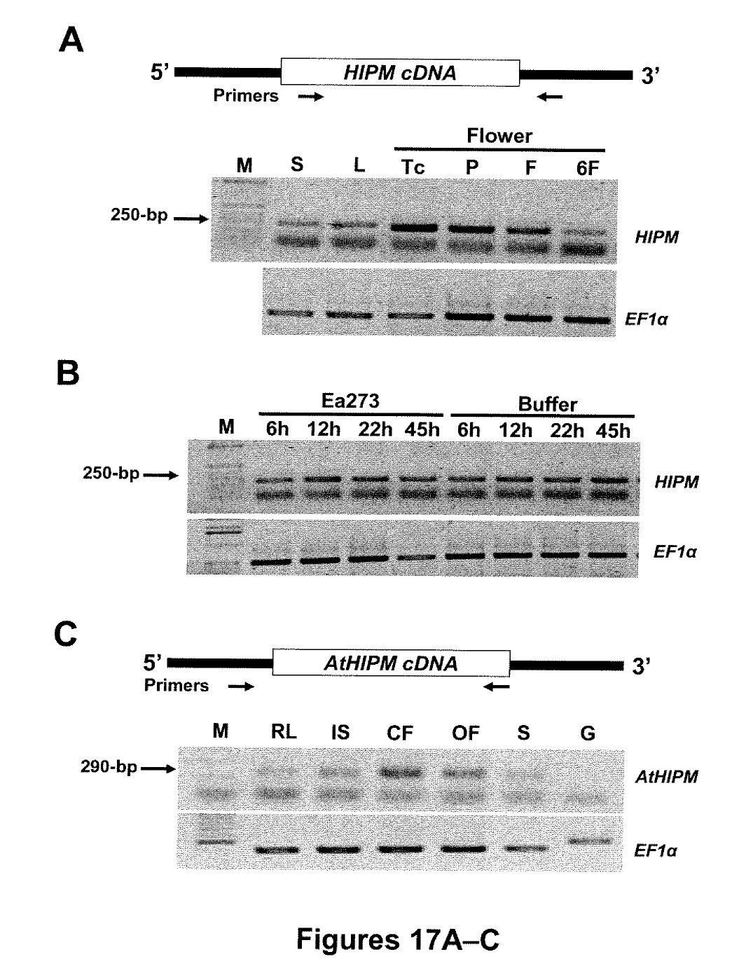

Patterns of expression of the HIPM gene were determined by northern hybridization using 10 μg samples of total RNA and a 250-bp fragment of HIPM cDNA as a probe. Because there were scant indications of HIPM expression, the more sensitive RT-PCR technique was used. HIPM transcripts were detected after more than 40 cycles of PCR using 700 ng of total RNA. Based on the RT-PCR results shown in FIG. 17A, expression of the HIPM gene is very low, and HIPM is expressed constitutively in leaves and shoots. However, HIPM is expressed more strongly in flowers than in leaves and shoots. Interestingly, HIPM expression was relatively strong in the TC, P, and F stages of flower development, but as petals started to fall, the expression level decreased to the same level as seen in leaves and shoots.

HIPM gene expression following inoculation of apple with E. amylovora strain Ea273 was also determined in leaves. Total RNA was isolated 6, 12, 22, and 45 hours after inoculating greenhouse-grown trees with E. amylovora or buffer. 700 ng of total RNA was used to determine the expression pattern of the HIPM gene by RT-PCR. As shown in FIG. 17B, the level of HIPM expression did not change as a result of inoculation with E. amylovora.

To determine the expression pattern of AtHIPM in Arabidopsis, total RNA isolated from leaves (“RL”), inflorescent shoots (“IS”), closed flowers (“CF”), open flowers (“OF”), and siliques (“S”) was analyzed by the same methods as used for HIPM from apple. As shown in FIG. 17C, like HIPM in apple, AtHIPM expression levels were low, and expression was strongest in closed and open flowers relative to other plant parts, as determined by RT-PCR with 1.7 μg of total RNA.

### Example 23

### AtHIPM Is Needed for HrpN to Enhance Growth of Arabidopsis

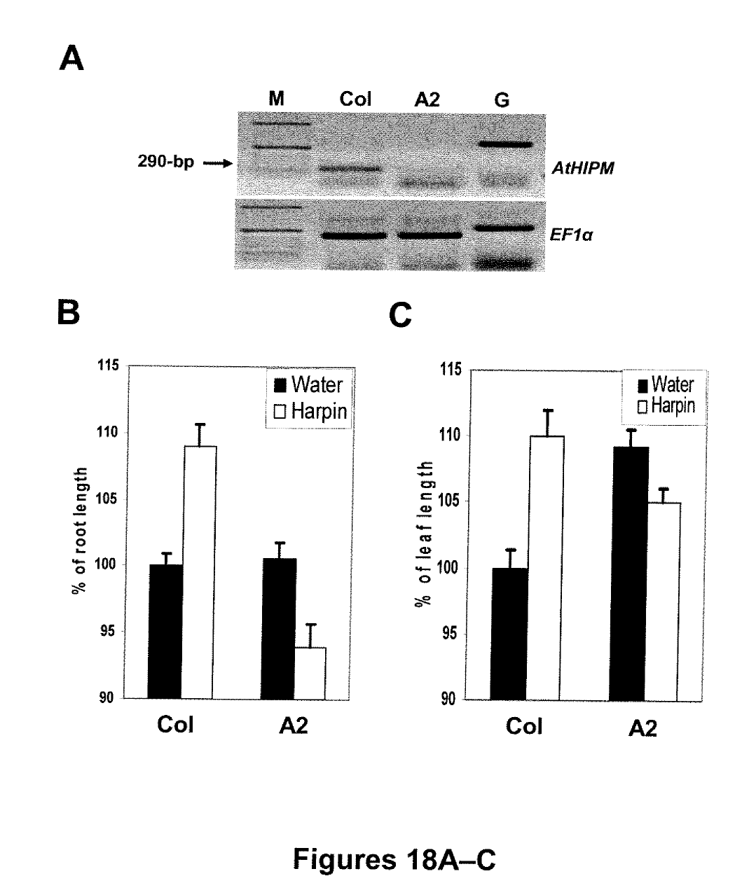

To determine the biological significance of the interaction of HrpN with HIPM and AtHIPM in plants, Arabidopsis was used because of the availability of its mutant lines, its short life cycle, and its ease of transformation. The effects of AtHIPM mutation on enhanced plant growth by HrpN were examined in Arabidopsis. One T-DNA insertion line was obtained from the Arabidopsis stock center (Columbus, Ohio, USA), in which T-DNA was inserted in the 5′-untranslated region (“UTR”) of the AtHIPM gene. Expression of AtHIPM was first tested in the mutant line by RT-PCR to ascertain whether the line would be useful for loss-of-function tests. As shown in FIG. 18A, few AtHIPM transcripts were present in the preparation from the T-DNA mutant relative to the wild-type, confirming its value for use in further experiments.

Under normal growing conditions, the mutant line of Arabidopsis grew like the wild-type-all developmental stages were normal. As shown in FIGS. 18B and 18C, however, the size of aerial parts (top growth) was around 9% larger than that of the wild-type plants, although root length did not differ significantly from that of the wild-type.

Treatment of Arabidopsis with HrpN resulted in beneficial effects. To determine the relationship between AtHIPM and the growth-enchancing activity of HrpN in Arabidopsis, the effect of HrpN on plant growth was examined in the mutant line compared to the wild-type Arabidopsis. First, root growth was determined in MS medium 10 days after treating both lines with 15 μg/ml of HrpN. As shown in FIG. 18B, root growth of the wild-type increased by around 9% after treatment with HrpN. However, in the mutant line, treatment with HrpN did not enhance plant growth. Instead, as shown in FIG. 18B, treatment with HrpN reduced root growth by around 6%. Secondly, the top growth of Arabidopsis was determined one week after spraying 3-week-old plants with 15 μg/ml of HrpN. Consistently, the top growth of the wild-type plants increased by around 10% following treatment with HrpN. However, as shown in FIG. 18C, treatment of the mutant line with HrpN reduced top growth. Interestingly, top growth of the mutant line that was treated with buffer was similar to that of the wild-type treated with HrpN. These results indicate that AtHIPM is needed for Arabidopsis to respond to HrpN treatment with enhanced growth.

### Example 24

### Overexpression of AtHIPM Reduces Plant Growth in Arabidopsis

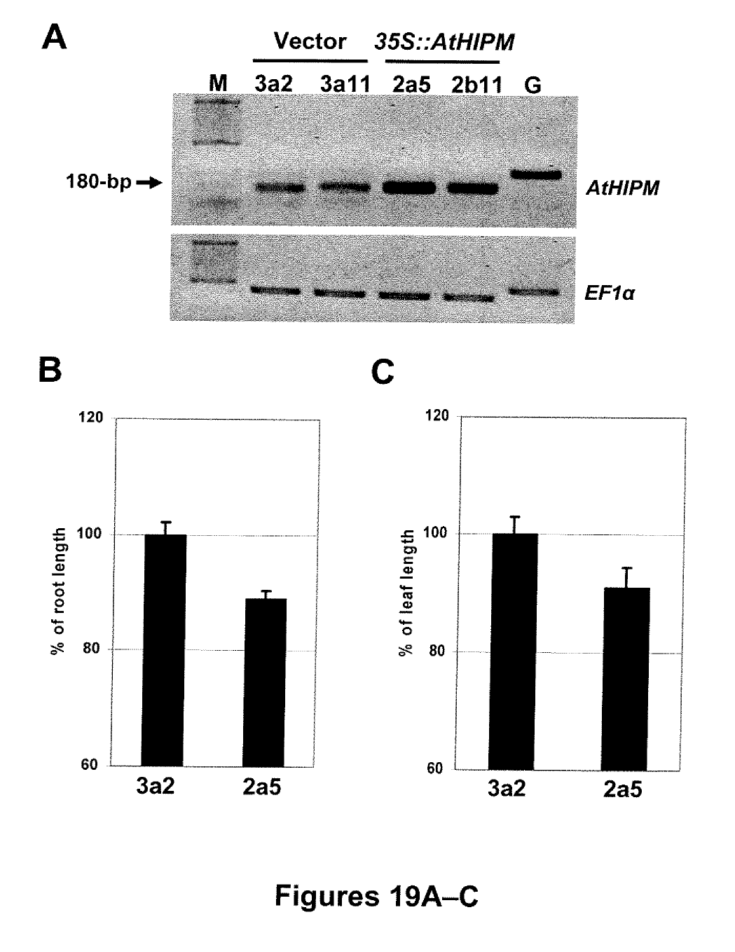

Two AtHIPM overexpressing lines and two vector-transformed lines were generated using vectors pBI121-AtHIPM and pBI121, respectively. The level of AtHIPM expression was first determined in these lines by RT-PCR, as shown in FIG. 19A. The level of AtHIPM expression in lines transformed with pBI121-AtHIPM was much higher than in lines transformed with pBI121. Root and top growth of these lines under normal growing conditions was examined. As shown in FIGS. 19B and 19C, both root and leaf length in AtHIPM overexpressing lines were smaller than in vector-transformed plants, indicating that AtHIPM functions as a negative regulator of plant growth. This finding is consistent with the evidence that AtHIPM mutation results in larger plants.

### Example 25

### Overexpression of the OsHIPM Gene in Rice

In Arabidopsis, overexpression of AtHIPM results in dwarfed plants, indicating that AtHIPM functions as a negative regulator of plant growth. To determine whether overexpression of OsHIPM results in the same phenotypes, lines that overexpress OsHIPM will be produced and tested for yield. It is expected that, as in Arabidopsis, overexpression of OsHIPM will result in dwarfed plants that produce less rice grain, or that “normal” yield will result from dwarfed plants (suggesting more efficient grain production).

### Example 26

### Mutation in AtHIPM Does not Disrupt Responses to 2,4-D, MeJA, and Ethylene in Arabidopsis

Since AtHIPM was shown to function as a negative regulator of plant growth, whether AtHIPM function is connected to the effects of several plant hormones that are involved in plant growth inhibition was examined. Both methyl jasmonate (“MeJA”) and auxin inhibit root growth in plants (Chadwick & Burg, “An Explanation of the Inhibition of Root Growth Caused by Indole-3-acetic Acid,” Plant Physiol. 42:415-20 (1967); Staswick et al., “Methyl Jasmonate Inhibition of Root Growth and Induction of a Leaf Protein are Decreased in an Arabidopsis thaliana Mutant,” Proc. Nat'I Acad. Sci. USA 89:6837-40 (1992), which are hereby incorporated by reference in their entirety). To determine whether AtHIPM is involved in MeJA- or auxin-mediated root growth inhibition, the root growth of the AtHIPM mutant line was measured in MS medium plates with 0, 1, or 5 μM of MeJA or 2,4-D. No difference in root growth inhibition was detected between the mutant line and the wild-type, although the inhibitory effects of MeJA and auxin were confirmed in both lines.

In the dark, treatment with ethylene causes a triple response in plants, characterized by swelling of the hypocotyls and inhibition of root and hypocotyl growth (Guzman & Ecker, “Exploiting the Triple Response of Arabidopsis to Identify Ethylene-related Mutants,” Plant Cell 2:513-23 (1990), which is hereby incorporated by reference in its entirety). To determine whether AtHIPM mutation affects the response to ethylene, the triple response in the AtHIPM mutant line was assessed in MS medium plates containing 10 μM of 1-aminocyclopropane-1-carboxylic acid (“ACC”), an ethylene precursor. The AtHIPM mutant line responded to ethylene just like the wild-type did.

### Discussion of Examples 1-26

HrpN-Interacting Proteins from Apple and Arabidopsis

HrpN-interacting proteins from apple were searched for using a yeast two-hybrid assay, and a single small protein, HIPM, was found. Orthologs of HIPM were found in Arabidopsis (AtHIPM) and the Nipponbare cultivar of rice (OsHIPM-N). HIPM and AtHIPM were studied, and evidence that their signal peptides are functional and that both proteins associate with plasma membranes of plant cells was found. Harpins, including HrpN of E. amylovora, are not translocated into plant cells, but they are secreted from bacteria and localize outside plant cells (Hoyos et al., “The Interaction of Harpin_{Pss}, with Plant Cell Walls,” Mol. Plant-Microbe Interact. 9:608-16 (1996); Perino et al., “Visualization of Harpin Secretion in Planta During Infection of Apple Seedlings by Erwinia amylovora,” Cell Microbiol. 1:131-41 (1999), which are hereby incorporated by reference in their entirety). The apoplastic location of HrpN and the localization of HIPM and AtHIPM, and possibly OsHIPM-N, to the plasma membrane suggests that interaction of HrpN with these proteins may occur in vivo.

In addition to demonstrating HIPM and AtHIPM interaction with HrpN, these proteins were shown to interact with HrpW, a second harpin of E. amylovora. Interaction of both HIPM and AtHIPM with both harpins suggests that HIPM and AtHIPM may be general interactors with harpins in plants. Because several other harpins have been characterized from other plant-pathogenic bacteria, it will be interesting to determine whether or not they also interact with HIPM or AtHIPM.

HIPM orthologs were found in three different plant species: apple, Arabidopsis, and rice. These plants represent diverse plant classes: two are dicots (one woody and one herbaceous) and one is a monocot. These findings suggest that the HIPM gene is conserved among plant species. However, the distribution of HIPM orthologs in many different plant species remains to be determined.

HrBP1 (HrpN-Binding Protein 1) from Arabidopsis

Researchers at Eden Bioscience Corporation reported finding HrBP1 (HrpN-binding protein 1) from Arabidopsis using a yeast two-hybrid assay with full-length HrpN as bait (U.S. Patent Publication No. 2004/0034554 to Shirley et al., which is hereby incorporated by reference in its entirety). HrBP1, which is quite different from HIPM, is 284 amino acids, and it exists and is expressed in many different plant species, including apple, based on northern hybridization with HrBP1 cDNA as a probe. However, its expression pattern and subcellular location are not known. Although researchers at Eden Bioscience found an HrBP1 homolog in apple by screening an apple cDNA library using the Arabidopsis HrBP1 gene as a probe, they did not report whether the apple HrBP1 interacts with HrpN in yeast or in vitro. Recently, another group working with HrBP1 found that apple HrBP1 did not interact with HrpN in yeast. Consistent with this finding, an apple homolog of HrBP1 was not detected during screening of the apple cDNA library described herein using HrpN as bait. These findings suggest that, unlike HIPM, HrBP1 may not be a target of HrpN protein in apple.

### Location of HIPM and AtHIPM Proteins in Plant Cells

HIPM and AtHIPM were shown to associate with plasma membranes of plant cells. Unlike other membrane proteins, HIPM and AtHIPM were not uniformly distributed in plasma membranes, but appeared to localize to some specific positions in plasma membranes. Furthermore, GFP signals fused with HIPM were coincident with plasma membranes under high osmotic conditions. This rules out the cell wall and plasmodesmata as possible target sites of HIPM and AtHIPM in plant cells.

Localization of HIPM and AtHIPM to some specific positions in plasma membranes could indicate that they are incorporated into lipid rafts. The presence of plasma membrane microdomains containing distinct molecular compositions such as lipid rafts has been reported mostly in mammalian cells, but recently also in plant cells (Bhat & Panstruga, “Lipid Rafts in Plants,” Planta 223:5-19 (2005), which is hereby incorporated by reference in its entirety). Lipid rafts exist in plasma membranes as microdomains consisting of several lipids, sterols, and integral and peripheral membrane proteins. In particular, these sites are enriched for many signaling molecules that regulate different signal transduction pathways such as endocytosis and exocytosis, apoptosis, and pathogen entry (Bhat & Panstruga, “Lipid Rafts in Plants,” Planta 223:5-19 (2005), which is hereby incorporated by reference in its entirety). Although little evidence has been reported, these sites seem to be a general target for pathogens to communicate with host cells (Rosenberger et al., “Microbial Pathogenesis: Lipid Rafts as Pathogen Portals,” Curr. Biol. 10:R823-5 (2000), which is hereby incorporated by reference in its entirety). Whether or not HIPM or AtHIPM is exclusively targeted to lipid rafts is unclear, but it would be very interesting to investigate this phenomenon.

### Expression of Both HIPM and AtHIPM in Flowers

Based on RT-PCR data, both HIPM and AtHIPM are weakly, constitutively expressed in leaves and stems. Interestingly, both genes are more strongly expressed in flowers than in leaves and stems of apple and Arabidopsis. Expression levels are reduced coincidently with the formation of fruiting structures. In apple, flowers are important infection sites for E. amylovora (FIRE BLIGHT (Joel L. Vanneste ed., 2000), which is hereby incorporated by reference in its entirety). Moreover, flowers are one of few fast growing tissues in plants. Because fast-growing tissues like flowers and shoot tips are the most susceptible parts to E. amylovora infection, higher expression of HIPM in flowers indicates that HrpN-HIPM interaction increases susceptibility to infection by stimulating the growth rate of plant cells in the infection sites.

The relationship between greater expression of HIPM in flowers and development of fire blight in apple has not been explored, but evaluation of HIPM-silenced apples currently under development may illuminate this relationship. It is predicted that the higher susceptibility of flowers to the initiation of fire blight infection may be related to the greater presence of HIPM in these tissues. Therefore, silencing expression of HIPM in apple will reduce the presence of HIPM in flowers and is expected to thereby reduce the susceptibility of flowers to the initiation of fire blight infection.

HrpN Domain for Interaction with HIPM

The 198 aa N-terminal region of HrpN was shown to be required for full interaction with HIPM. This portion of HrpN, HrpN¹⁻¹⁹⁸, includes both the defense domain (HrpN¹⁻¹⁰⁴) and the growth domain (HrpN¹³⁷⁻¹⁸⁰), which are responsible, respectively, for induction of defense responses and growth promotion in plants. The fact that the same region of HrpN that is involved in its interaction with HIPM and AtHIPM is necessary for enhanced growth in response to HrpN in plants is indicative of the biological importance of HIPM as a HrpN-interacting protein.

Although the 198 aa N-terminal region of HrpN was sufficient for interaction with HIPM, it was found to not be sufficient for virulence in immature pear fruits. This suggests that virulence requires portions of the C-terminus of HrpN in addition to the 198 aa N-terminal region necessary for interaction with HIPM. It is not clear how the N-terminal portion and the C-terminal portion of HrpN function together for virulence of E. amylovora, but two hypothetical models are proposed. After HIPM interacts with the N-terminal portion of HrpN, the C-terminal portion of HrpN may block interaction of HIPM with a secondary host protein, which may be located in plasma membranes (like receptor kinases). Under normal conditions, interaction of HIPM and a secondary host protein may increase disease resistance, but if HrpN is present, this interaction may be interrupted, resulting in inhibition of defense responses. Alternatively, interaction of the N-terminal portion of HrpN may allow the C-terminal portion of HrpN to interact with a second host protein that has no physical interaction with HIPM. This interaction may block a positive regulator function of a second host protein in disease resistance.

### Significance of the Presence of AtHIPM in Arabidopsis in its Response to HrpN

HrpN is required for development of the fire blight disease in apple, and it also induces several beneficial effects in plants, such as growth enhancement. As shown herein, top growth of an AtHIPM mutant line treated with buffer was similar to the growth of the wild-type plant treated with HrpN. In addition, overexpression of AtHIPM resulted in smaller plants. These observations indicate that AtHIPM acts as a negative regulator of plant growth. A negative regulatory function of AtHIPM may explain why plants exhibit enhanced growth after treatment with HrpN-when HrpN is present, it may intercept AtHIPM by protein-protein interaction, resulting in the inhibition of its negative regulatory function. This inhibition may lead to larger plants, as does mutation of AtHIPM.

Without being bound by theory, it is proposed herein that HrpN-HIPM interaction increases susceptibility to E. amylovora by controlling the growth rate of plant cells in the infection sites. This conclusion is based on the growth-enhancing activity of HrpN, higher expression of the HIPM and AtHIPM genes in fast-growing susceptible tissues like flowers, and the function of AtHIPM as a negative regulator of plant growth. In stems and leaves of non-host plants like Arabidopsis and rice, treatment with HrpN may block a negative regulatory function of AtHIPM and/or OsHIPM by protein-protein interaction, resulting in enhanced growth. In addition, in flowers of host plants like apple, E. amylovora secretes HrpN protein that may block a negative regulator function of HIPM to increase growth rate, resulting in an increase in susceptibility at the infection sites. Thus, growth-enhancing activity of HrpN is probably related to its virulence activity in host plants by blocking HIPM function.

Interestingly, treatment of the AtHIPM mutant line with HrpN reduced plant growth as compared to the water-treated control. This indicates that HrpN itself may inhibit growth of plant cells in the absence of AtHIPM. Previously, HrpN was shown to cause ion leakage by regulating ion channels in Arabidopsis suspension cells (El-Maarouf et al., “Harpin, a Hypersensitive Response Elicitor from Erwinia amylovora, Regulates Ion Channel Activities in Arabidopsis thaliana Suspension Cells,” FEBS Lett. 497:82-4 (2001), which is hereby incorporated by reference in its entirety), and other harpins, HrpZ and PopA, have pore-forming activity (Lee et al., “HrpZ(Psph) from the Plant Pathogen Pseudomonas syringae pv. phaseolicola Binds to Lipid Bilayers and Forms an Ion-conducting Pore In Vitro,” Proc. Nat'l Acad. Sci. USA 98:289-94 (2001); Racape et al., “Ca²⁺-dependent Lipid Binding and Membrane Integration of PopA, a Harpin-like Elicitor of the Hypersensitive Response in Tobacco,” Mol. Microbiol. 58:1406-20 (2005), which are hereby incorporated by reference in their entirety). It has been shown herein that HrpN interacts with itself in yeast, suggesting that the protein might be present in multimeric forms, as was suggested for HrpZ_{Pss} (Chen et al., “An Amphipathic Protein from Sweet Pepper Can Dissociate Harpin_{Pss} Multimeric Forms and Intensify the Harpin_{Pss}-mediated Hypersensitive Response,” Physiol. Mol. Plant Pathol. 52:139-49 (1998), which is hereby incorporated by reference in its entirety). This suggests that ion leakage or possible pore-forming activity may be related to the negative growth effect caused by HrpN. Interestingly, HRAP (hypersensitive response-assisting protein), which intensifies the HrpZ_{Pss}-mediated hypersensitive response in sweet pepper, is an amphipathic protein that can dissociate multimeric forms of HrpZ_{Pss} into monomeric or dimeric forms (Chen et al., “An Amphipathic Protein from Sweet Pepper Can Dissociate Harpin_{Pss} Multimeric Forms and Intensify the Harpin_{Pss}-mediated Hypersensitive Response,” Physiol. Mol. Plant Pathol. 52:139-49 (1998); Chen et al., “cDNA Cloning and Characterization of a Plant Protein That May be Associated with the Harpin_{PSS}-mediated Hypersensitive Response,” Plant Mol. Biol. 43:429-38 (2000, which are hereby incorporated by reference in their entirety). Although HRAP is radically different from HIPM or AtHIPM, HIPM and AtHIPM might have similar functions as HRAP. If they act like HRAP in vivo, they may dissociate multimeric forms of HrpN into monomers or dimers. Although no evidence has been reported that HrpN is present in multimeric forms in vivo or how HrpN triggers ion leakage in Arabidopsis suspension cultures, formation of mutimers of HrpN may be necessary, because several molecules of HrpN are needed for formation of pores in the lipid bilayer. Thus, without HIPM or AtHIPM, a multimeric form of HrpN may trigger ion leakage or form pores in plasma membranes, which results in negative effects on plant cells.

The present findings that HrpN interacts with HIPM and AtHIPM and that AtHIPM functions as a negative regulator of plant growth shed light on how HrpN contributes to the development of fire blight disease in host plants. Apple transgenic plants, in which the HIPM gene is silenced, will provide more direct evidence as to whether or not HIPM is important for the development of fire blight disease. In addition, the present findings provide some clues about how growth-enhancing activity of HrpN can be connected to its virulence activity. This connection could be confirmed if site-directed mutants of HrpN protein lacking growth-enhancing activity were discovered. It would be intriguing to investigate whether HrpN mutant proteins without growth-enhancing activity still contain virulence activity by determining whether those proteins restore virulence of E. amylovora hrpN mutants in host plants.

### Possible Roles of HIPM, AtHIPM, and OsHIPM in Plants

In shoot meristems, CLAVATA proteins (CLV1, 2, and 3) control proliferation and differentiation of meristems (Clark et al., “The CLAVATA and SHOOT MERISTEMLESS Loci Competitively Regulate Meristem Activity in Arabidopsis,” Development 122:1567-75 (1996), which is hereby incorporated by reference in its entirety). CLV3 is a small extracellular protein that interacts in plasma membranes with CLV1, a receptor protein kinase, which results in coordination of meristem cell growth (Trotochaud et al., “CLAVATA3, a Multimeric Ligand for the CLAVATA1 Receptor-kinase,” Science 289:613-7 (2000); Rojo et al., “CLV3 Is Localized to the Extracellular Space, Where it Activates the Arabidopsis CLAVATA Stem Cell Signaling Pathway,” Plant Cell 14:969-77 (2002), which are hereby incorporated by reference in their entirety).

Both HIPM and AtHIPM are small proteins like CLV3, and they associate with plasma membranes. Under normal conditions, HIPM and AtHIPM may interact with other proteins in plasma membranes, like CLV1, to regulate plant growth in a negative manner. Based on a GFP targeting experiment, both HIPM and AtHIPM were shown to exist in clusters in plasma membranes, suggesting that they are targeted to some specific positions in plasma membranes. Expression patterns of both HIPM and AtHIPM indicate that they are more strongly expressed in fast-growing tissues like flowers and shoot tips than in relatively slow-growing tissues like leaves and stems. However, HIPM and AtHIPM might function not as a positive modulator, but as a negative one, because mutation of AtHIPM results in increased growth.

Interestingly, the OsHIPM ortholog in the Jefferson cultivar lacks a start codon, suggesting that it is non-functional. Comparison of the Jefferson and Nipponbare cultivars in terms of plant size reveals that the Nipponbare cultivar is much larger. If OsHIPM is a critical factor determining plant size, other genetic differences also must affect size, as the size differences observed are the natural phenotype opposite to that seen in Arabidopsis. In addition, AtHIPM does not seem to be a major factor determining plant size in Arabidopsis, but it seems to fine-tune plant growth. Therefore, OsHIPM-N silencing or overexpression may affect plant growth and/or grain yield in the Nipponbare cultivar like AtHIPM affects plant size in Arabidopsis.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A nucleic acid molecule configured to increase or decrease expression of a nucleic acid molecule that encodes a HrpN-interacting protein, wherein the HrpN-interacting protein is selected from the group consisting of (i) a protein having an amino acid sequence selected from the group consisting of SEQ ID NO: 2, SEQ ID NO: 4, and SEQ ID NO: 6; (ii) a protein encoded by a nucleotide sequence selected from the group consisting of SEQ ID NO: 1, SEQ ID NO: 3, and SEQ ID NO: 5; and (iii) a protein at least 90% homologous and/or identical to the protein of (i) or (ii).

2. The nucleic acid molecule according to claim 1, wherein the nucleic acid molecule is configured to increase expression of the HrpN-interacting protein.

3. The nucleic acid molecule according to claim 2, wherein the nucleic acid molecule comprises a nucleotide sequence selected from the group consisting of SEQ ID NO: 1, SEQ ID NO: 3, SEQ ID NO: 5, SEQ ID NO: 31, SEQ ID NO: 32, and SEQ ID NO: 33.

4. The nucleic acid molecule according to claim 1, wherein the nucleic acid molecule is configured to decrease expression of the HrpN-interacting protein.

5. The nucleic acid molecule according to claim 4, wherein the nucleic acid molecule comprises a nucleotide sequence selected from the group consisting of SEQ ID NO: 27 or a fragment thereof at least about 20 nucleotides in length, SEQ ID NO: 28 or a fragment thereof at least about 20 nucleotides in length, SEQ ID NO: 29 or a fragment thereof at least about 20 nucleotides in length, and SEQ ID NO: 30 or a fragment thereof at least about 20 nucleotides in length.

6. A nucleic acid construct comprising:
a nucleic acid molecule according to claim 1;
a 5′ promoter sequence; and
a 3′ terminator sequence, wherein the nucleic acid molecule, the promoter sequence, and the terminator sequence are operatively coupled to permit transcription of the nucleic acid molecule.

7. The nucleic acid construct according to claim 6, wherein the nucleic acid molecule is configured to increase expression of the HrpN-interacting protein.

8. The nucleic acid construct according to claim 7, wherein the nucleic acid molecule encodes the HrpN-interacting protein and is in sense orientation.

9. The nucleic acid construct according to claim 7, wherein the nucleic acid molecule comprises a nucleotide sequence selected from the group consisting of SEQ ID NO: 1, SEQ ID NO: 3, SEQ ID NO: 5, SEQ ID NO: 31, SEQ ID NO: 32, and SEQ ID NO: 33.

10. The nucleic acid construct according to claim 6, wherein the nucleic acid molecule is configured to decrease expression of the HrpN-interacting protein.

11. The nucleic acid construct according to claim 10, wherein the nucleic acid molecule is positioned in the nucleic acid construct to result in suppression or interference of endogenous mRNA encoding the HrpN-interacting protein.

12. The nucleic acid construct according to claim 10, wherein the nucleic acid molecule is an antisense form of at least a portion of a HrpN-interacting protein-encoding nucleic acid molecule.

13. The nucleic acid construct according to claim 10, wherein the nucleic acid molecule comprises a first segment encoding at least a portion of a HrpN-interacting protein, a second segment in an antisense form of the first segment, and a third segment linking the first and second segments.

14. The nucleic acid construct according to claim 10, wherein the nucleic acid molecule comprises a nucleotide sequence selected from the group consisting of SEQ ID NO: 27 or a fragment thereof at least about 20 nucleotides in length, SEQ ID NO: 28 or a fragment thereof at least about 20 nucleotides in length, SEQ ID NO: 29 or a fragment thereof at least about 20 nucleotides in length, and SEQ ID NO: 30 or a fragment thereof at least about 20 nucleotides in length.

15. An expression vector comprising the nucleic acid according to claim 1.

16. A host cell transformed with the nucleic acid construct according to claim 6.

17. A plant transformed with the nucleic acid construct according to claim 6.

18. A plant seed produced from the plant according to claim 17.

19. A plant seed transformed with the nucleic acid construct according to claim 6.

20. A method of increasing or decreasing plant growth, said method comprising:
providing a transgenic plant or plant seed transformed with a nucleic acid construct according to claim 6 and
growing the transgenic plant or a transgenic plant grown from the transgenic plant seed under conditions effective to increase or decrease plant growth compared to non-transgenic plants.

21. The method according to claim 20, wherein said increase in plant growth is characterized by greater yield, increased quantity of seeds produced, increased percentage of seeds germinated, increased plant size, increased plant height, increased root growth, increased leaf growth, greater biomass, more and bigger fruit, earlier germination, earlier fruit and/or plant coloration, and earlier fruit and/or plant maturation.

22. A method of imparting disease resistance to plants, said method comprising:
providing a transgenic plant or plant seed transformed with a nucleic acid construct according to claim 6 and
growing the transgenic plant or a transgenic plant grown from the transgenic plant seed under conditions effective to impart disease resistance to the plant compared to non-transgenic plants.

23. The method according to claim 22, wherein the disease is fire blight.

24. An isolated nucleic acid molecule comprising bases 90 to 269 of the nucleotide sequence of SEQ ID NO: 1.

25. An isolated protein or polypeptide comprising the amino acid sequence of SEQ ID NO: 2.
